(19) United States
(12) Patent Application Publication
(10) Pub. No.: US 2026/0237474 A1
(43) Pub. Date: Aug. 13, 2026

(54) IMAGE ANALYTICS FOR CHARTING

(71) Applicant: ZOLL Medical Corporation, Chelmsford, MA (US)

(72) Inventors: Keenan S. Early, Denver, CO (US); Guy Johnson, Wilton, NH (US); Gary A. Freeman, Waltham, MA (US); Frederick W. Forester, Encinitas, CA (US); Peter G. Goutmann, Gibsonia, PA (US)

(73) Assignee: ZOLL Medical Corporation, Chelmsford, MA (US)

(21) Appl. No.: 19/469,620

(22) PCT Filed: Mar. 27, 2024
(86) PCT No.: PCT/US2024/021734

§ 371 Date: Sep. 26, 2025

Related U.S. Application Data
(60) Provisional application No. 63/455,897, filed on Mar. 30, 2023.

(51) Int. Cl. G16H 10/60; G06V 10/82; G16H 20/40

(52) U.S. Cl. CPC G16H 10/60; G06V 10/82; G16H 20/40

(57) ABSTRACT

In an illustrative embodiment, a patient data charting apparatus for automatically populating electronic patient care record (ePCR) data at an emergency medical scene includes processor(s) configured to obtain image data image capture device(s), the image data including a sequence of images of performance of a medical procedure by at least one emergency medical services (EMS) caregiver, analyze the image data to identify a sequence of steps corresponding to medical procedure(s), where, for each of at least a portion of the steps, analyzing includes recognizing, within the image data, at least one medical equipment item used. The processor(s) may be configured to identify the medical procedure, determine, based at least in part on the medical procedure, value(s) of data field(s), and populate data field(s) of the ePCR with the value(s).

### RELATED APPLICATIONS

This application is a national phase filing of PCT Application No. PCT/US2024/021734 entitled “Image Analytics for Charting and filed Mar. 27, 2024, which claims priority to U.S. Provisional Patent Application No. 63/455,897 entitled “Image Analytics for Charting” and filed Mar. 30, 2023. The contents of each above-noted application are hereby incorporated by reference in its entirety.

### BACKGROUND

Emergency medical services (EMS) agencies create and use an electronic patient care record (ePCR) for each patient encounter. The ePCR contains a complete record of medical observations and treatments for the patient during the patient encounter. The ePCR includes times for the observations and treatments, patient medical history information, and transport information (e.g., from a scene of an emergency to a medical care facility). Based in part on the complexities of medical diagnosis and care in these situations along with governmental reporting guidelines, the ePCR may be typically a complex and lengthy document.

Software applications exist that interact with EMS personnel to complete ePCRs. These software applications may include user interface screens with controls to receive input from EMS personnel regarding a patient encounter in the pre-hospital setting. This input specifies values of data fields that document the complete record of medical observations and treatments described above. EMS personnel need to attend to the entry of information into the ePCR while also making quick decisions about interventions for emergency conditions such as respiratory distress, cardiac arrest, trauma, drug overdose, etc. In the pre-hospital setting, these decisions must often be made with little to no information about a patient's medical history.

### SUMMARY OF ILLUSTRATIVE EMBODIMENTS

In one aspect, the present disclosure relates to a patient data charting apparatus for automatically populating electronic patient care record (ePCR) data at an emergency medical scene, the patient data charting apparatus including a memory configured to store an ePCR including a number of data fields, and at least one processor. The at least one processor may be configured to obtain image data from at least one image capture device, where the image data includes a sequence of images of performance of a medical procedure by at least one emergency medical services (EMS) caregiver, analyze the image data to identify a sequence of steps corresponding to one or more medical procedures, where, for each respective step of at least a portion of the set of steps, the analyzing includes recognizing, within the image data, at least one medical equipment item used during the respective step, identify, based at least in part on the set of steps, the medical procedure, responsive to the identification, determine, based at least in part on the medical procedure, at least one first value of at least one first data field of the number of data fields, and populate the at least one first data field of the ePCR with the at least one first value.

In some embodiments, the at least one processor is further configured to analyze an initial one or more images of the sequence of images to recognize a precursor step performed prior to beginning the medical procedure. The precursor step may include preparing a given medical equipment item of the at least one medical equipment item. The precursor step may include preparing a site on the patient's body.

In some embodiments, recognizing the at least one medical equipment item includes recognizing an identification marking on a given medical equipment item of the at least one medical equipment item. The identification marking may include a particular shape and/or a particular color assigned to the given medical equipment item. The identification marking may include a machine-readable code.

In some embodiments, the at least one medical equipment item includes one or more of gloves, a 3-lead electrocardiogram (EKG), a 12-lead EKG, a cardiac monitor, a cardioverter, a central intravenous (IV) catheter, an IV bag, a defibrillator, tubing, a ventilator, a bag valve mask, a tourniquet, a splint, a backboard, a cervical collar, a gurney, gauze, an alcohol swab, or a nasal cannula. The set of medical procedures may include one or more of oxygen delivery, intravenous saline delivery, intravenous drug delivery, obtaining a set of vital physiological measurements, cricothyrotomy, nasal intubation, endotracheal intubation, or tourniquet application with infusion.

In some embodiments, the patient data charting apparatus includes one or more image capture devices of the at least one image capture device. The at least one image capture device may include a light detection and ranging (LiDAR) device. The at least one image capture device may include a video image capture device. The video image capture device may be a wearable device. The video image capture device may include a smart glasses device, a smart watch device, or a body camera.

In some embodiments, the at least one image capture device is configured to capture three-dimensional image data. One or more image capture devices of the at least one image capture device may be mounted in or on a medical transport vehicle. One or more image capture devices of the at least one image capture device may be configured to be held or worn by a caregiver.

In some embodiments, one or more image capture devices of the at least one image capture device are mounted to or integrated into medical equipment. A first image capture device of the one or more image capture devices may be mounted to or integrated into a gurney. One or more image capture devices of the at least one image capture device may be mounted to or integrated into a remotely controlled movable mount. The remotely controlled movable mount may be a drone. The remotely controlled movable mount may be configured to track a position of a patient or a caregiver.

In some embodiments, the at least one image capture device is configured to activate image capture based on a detection of an emergency scene activity. The at least one image capture device may be configured to begin capturing the image data responsive to motion detection. The at least one image capture device may be configured to begin capturing the image data responsive to detection of an audible command. The at least one processor may be further configured to activate the at least one image capture device responsive to detecting presence of a patient. Detecting the presence of the patient may include detecting, via sensor signals, the patient disposed on a treatment or transportation surface. A gurney may include the treatment or transportation surface. The at least one processor may be further configured to activate the at least one image capture device responsive to detecting arrival at a medical emergency scene. Detecting the arrival may include analyzing global positioning system (GPS) signals.

In some embodiments, a patient data charting device includes the memory and one or more processors of the at least one processor. The patient data charting device may be embodied in one or more of a smartphone, a tablet, a portable computing device, a wearable computing device, or combinations thereof.

In some embodiments, the patient data charting apparatus further includes an artificial intelligence (AI) analytics processor system, and a set of AI models, each AI model corresponding to a given procedure of the set of procedures. Analyzing the image data may include processing, by the AI analytics processor system, the image data in view of at least a portion of the set of procedures. Analyzing the image data may include applying the image data to at least a portion of the set of AI models. The set of AI models may include a first subset configured to analyze a first type of a number of types of image data and a second subset configured to analyze a second type of the number of types of image data. The number of types of image data may include one or more of full color data, black and white data, wireframe data, LiDAR data, or three-dimensional image data. One or more AI models of the set of AI models may be configured to analyze image data captured by a number of cameras. The at least one processor may be further configured to convert the image data to a format compatible with at least a portion of the set of AI models. Converting the image data may include co-registering a number of image data sets obtained by two or more image capture devices of the at least one image capture device. Converting the image data may include reducing a resolution of the image data. The format may be a wireframe format.

In some embodiments, an organizational structure of the ePCR is defined in at least one ePCR standard. The at least one ePCR standard may be a National Emergency Medical Service Information System (NEMSIS) standard. An organizational structure of the ePCR may include data field sections organized according to medical procedure categories. The data field sections may include one or more of a respiratory section or a cardiac section.

In some embodiments, the at least one processor is further configured to identify at least one second data field of the number of data fields as being procedurally related to the at least one first data field, and identify, based on the image data, at least one second value of the at least one second data field. The procedural relationship may correspond to a relationship between steps in a treatment procedure. The procedural relationship may correspond to a treatment protocol. The treatment protocol may be defined within an intervention sequence of activities and/or data entry. The at least one processor may be configured to identify the at least one second data field as being procedurally related to the at least one first data field based on a predictive workflow. The predictive workflow may identify procedurally related fields based on one or more of a geolocation of an EMS transport mode, a type of EMS service, or a medical protocol.

In some embodiments, the at least one processor is further configured to determine one or more of patient medical information or patient demographic information from the image data. The medical information may include one or more of an identifier of a medication from a medication label, electrocardiogram (ECG) information from an ECG tape, ECG information from a screen shot of a medical device display, or patient physiological information from a screen shot of a medical device display. The patient demographic information may include one or more of driver's license information, insurance card information, or patient information from a face sheet. Determining the one or more of the patient medical information or the patient demographic information may include recognizing and analyzing handwritten text. Determining the patient medical information may include processing portions of the image data including at least a partial view of a patient to derive physiological metrics from analyzing the patient as captured in the image data.

In some embodiments, chest movements of the patient are analyzed to derive patient breathing metrics. The at least one image capture device may include a thermal imaging device and/or an infrared imaging device. Deriving physiological metrics from analyzing the patient may include deriving temperature, pulse, and/or respiration rate of the patient from spectral analysis of non-visible spectrum image data.

In some embodiments, the at least one processor is further configured to identify, by analyzing the image data, at least one caregiver. Analyzing the image data may include identifying, within the image data, a caregiver identification badge. Analyzing the image data may include performing facial recognition on a portion of the image data to obtain facial recognition metrics, and matching the facial recognition metrics to a particular caregiver of a number of caregivers. Recognizing the medical procedure may include recognizing performance of at least a portion of a set of steps of the medical procedure by a particular caregiver of the at least one caregiver. The at least one processor may be further configured to determine, using an identification of the particular caregiver, a certification level, an employee level, and/or skill level of the particular caregiver. Recognizing the medical procedure may include matching the medical procedure to the certification level, the employee level, and/or the skill level of the particular caregiver. The number of caregivers may include one or more of an emergency medical technician, a paramedic, a medic, a physician, a nurse, or a medical scribe.

In some embodiments, the at least one processor is further configured to obtain, from one or more devices, physiological data including metric values and/or sensor data corresponding to one or more physiological metrics of the patient. The one or more physiological metrics may include at least one of heart rate, respiration rate, blood pressure, or temperature. The one or more physiological metrics may include at least one of EKG metrics or ECG metrics. The one or more devices may include a medical monitoring device. The one or more devices may include a portable computing device.

In some embodiments, the at least one processor is further configured to obtain, from one or more devices, alarm information indicative of at least one warning or error related to a medical procedure. The alarm information may include at least one of a ventilator alarm, a bag valve mask sensor alarm, an impedance sensor alarm, an airflow sensor alarm.

In some embodiments, the at least one image capture device includes at least one LiDAR sensor configured to collect LiDAR sensor data, and the at least one processor is further configured to generate, based at least in part on the LiDAR sensor data, an object map of a section of the emergency medical scene including a number of objects and associate each object of at least a portion of the number of objects with an identifier. The at least one processor may be further configured to analyze movements in the LiDAR sensor data captured by the at least one LiDAR sensor to identify activity indicative of preparation for the medical procedure. Identifying the activity indicative of the preparation for the medical procedure may include recognizing the identifier associated with at least one object of the one or more objects corresponds to the medical procedure. The at least one image capture device may include at least one camera device, and generating the object map may include aligning the LiDAR sensor data and camera data captured by at least one camera to identify at least a subset of the number of objects. The section of the emergency medical care scene may be a section of an interior of an emergency medical vehicle. The at least one processor may be further configured to generate an equipment inventory for the emergency medical vehicle based on the identifiers of the at least the portion of the number of objects. The LiDAR sensor data may be wireframe data.

In some embodiments, the patient data charting apparatus further includes a network interface coupled to the at least one processor and configured to communicably couple to at least one separate computing device via a network. The at least one separate computing device may include a first image capture device of the at least one image capture device. The at least one separate computing device may include an edge server. The edge server may be configured to perform at least a portion of the operations. The edge server may be configured to analyze the image data to identify the set of steps. The apparatus may include the edge server. The edge server may include a portion of the at least one processor.

In some embodiments, the at least one processor is further configured to determine, responsive to analyzing the image data, at least one of success or failure of the medical procedure. Determining the at least one of success or failure of the medical procedure may include calculating at least one timing of the medical procedure. A first timing of the at least one timing may correspond to a length of time maintaining a piece of medical equipment in a position of therapeutic use. Determining the at least one of success or failure of the medical procedure may include recognizing removal of at least one piece of medical equipment. Determining the at least one of success or failure of the medical procedure may include referencing physiological data of the patient obtained contemporaneously with and/or subsequent to performance of the medical procedure. Determining the at least one of success or failure of the medical procedure may include recognizing an end point of the set of steps. Determining the at least one of success or failure of the medical procedure may include recognizing repetition of at least a portion of the set of steps.

In one aspect, the present disclosure relates to a patient data capture apparatus for automatically identifying patient care at an emergency medical scene, the patient data capture apparatus including a memory storing an ePCR including a number of data fields, and at least one processor configured to obtain image data from the at least one image capture device, where the image data includes a sequence of images of performance of a medical procedure by an emergency medical services (EMS) caregiver, analyze the image data to identify actions corresponding to one or more medical procedures, analyze the image data to identify contextual information corresponding to at least one of the one or more medical procedures, the contextual information including one or more of medical equipment used during the performance of the medical procedure, a physiological data pattern of the patient during the performance of the medical procedure, or a skill level of at least one caregiver involved in the performance of the medical procedure, identify, based at least in part on the actions and the contextual information, the medical procedure, responsive to the identification, select, based at least in part on the medical procedure, a billing category corresponding to the medical procedure from a number of available billing categories, and populate at least one data field of the ePCR based on the identified action and the billing category.

In some embodiments, the patient data capture apparatus further includes the at least one image capture device. The patient data capture apparatus may further include a non-volatile computer readable memory storing a number of billing codes corresponding to a number of medical procedures, where each medical procedure of at least a portion of the number of medical procedures is correlated to a billing category of the number of billing categories. The at least one processor may be further configured to match the medical procedure and the billing category to a billing code.

In some embodiments, the number of billing categories includes basic life support (BLS), advanced life support (ALS), ALS level 1 (ALS1), and ALS level 2 (ALS2). The one or more medical procedures may include one or more of 3-Lead EKG, 12-Lead EKG, advanced life support (ALS) assessment, cardiac monitor, cardioversion, cardiac pacing, central intravenous (IV), central venous line, chest decompression, chest tube, combitube, cricothyrotomy, needle cricothyrotomy, defibrillation, manual defibrillation/cardioversion, external pacing, endotracheal intubation, urinary catheter, internal pacing, Intraosseous (IO) access, IO line, nasotracheal intubation, needle thoracotomy, needle thoracostomy, nasogastric tube, peripheral IV, orogastric tube, orotracheal, SIO, surgical airway, or surgical cricothyroidotomy.

In some embodiments, identifying the skill level of the at least one caregiver includes identifying, within the image data, a caregiver identification badge. The at least one processor may be further configured to determine, using identification information of the identification badge, one or more of a certification level, an employee level, or a skill level of the particular caregiver. Identifying the skill level of the at least one caregiver may include performing facial recognition on a portion of the image data to obtain facial recognition metrics, and matching the facial recognition metrics to a particular caregiver of a number of caregivers.

In some embodiments, identifying the medical equipment includes recognizing an identification marking on a given medical equipment item of at least one medical equipment item. The identification marking may include a particular shape and/or a particular color assigned to the given medical equipment item. The identification marking may include a machine-readable code.

In some embodiments, identifying the physiological data pattern includes recognizing, in the image data, at least one of electrocardiogram (ECG) information from an ECG tape and/or a screen shot of a medical device display, or patient physiological information from a screen shot of a medical device display. The at least one processor may be further configured to obtain, from one or more devices, physiological data including metric values and/or sensor data corresponding to one or more physiological metrics of the patient. Identifying the physiological data pattern may include analyzing the physiological data.

In some embodiments, the patient data capture apparatus further includes a set of machine learning models, each machine learning model corresponding to a given procedure of the one or more medical procedures. Analyzing the image data may include applying at least a portion of the set of machine learning models to the image data. Each machine learning model of the set of machine learning models may be trained to identify a respective activity workflow of a number of activity workflows corresponding to the given procedure based on a time series of image data. Each activity workflow of the number of activity workflows may include a respective set of steps. For each respective machine learning model of at least a portion of the set of machine learning models, the activity workflow of the respective machine learning model may be associated with at least one of a respective precursor step of a set of precursor steps preceding the activity workflow or a respective completion step of a set of completion steps subsequent to the activity workflow. Analyzing the image data may include recognizing, in an initial portion of the image data, a given precursor step of the set of precursor steps, and selecting, based on the given precursor step, the portion of the set of machine learning models to apply to the image data. The at least one processor may be further configured to archive sets of image data for updating the training of at least a portion of the machine learning models. Archiving the sets of image data may include obscuring identifying features that could be used to identify a patient. The identifying features may include one or more of facial features, tattoos, medical bracelet information, or medical chart information visible in the image data. The at least one processor may be further configured to archive, in correlation with each of the sets of image data, one or more procedure codes and/or billing codes entered in association with care of a patient at the medical scene. The at least one processor may be further configured to access one or more procedure codes and/or billing codes entered in association with care of a patient at the medical scene, and review the one or more procedure codes and/or billing codes for a match to the medical procedure. The at least one processor may be further configured to, responsive to succeeding in identifying the match to the medical procedure, label the corresponding archived set of image data as positive training data. The at least one processor may be further configured to, responsive to failing to identify the match to the medical procedure, label the corresponding archived set of image data as negative training data. The at least one processor may be further configured to archive, in correlation with each of the sets of image data, the contextual data.

In some embodiments, the at least one processor is further configured to obtain, via a network from a separate computing device, additional context data. The patient data capture apparatus may further include processing circuitry for training machine learning classifiers including the set machine learning models, the processing circuitry configured to access the archived sets of image data and correlated contextual data, and train a set of context-aware machine learning models, each context-aware machine learning model of the set of context-aware machine learning models configured to recognize a respective medical procedure of the set of medical procedures by analyzing captured image data in view of corresponding additional context data. The additional context data may include one or more of alarm information from one or more medical devices, proximity data derived from at least one of near-field wireless communications or short-range wireless communications of the one or more medical devices, or an energy signature of one or more medical devices. The additional context data may include at least one of identification data or proximity data obtained from one or more nearby computing devices. The additional context data may include ePCR data.

In some embodiments, the set of machine learning models include the set of context-aware machine learning models, such that training the set of context-aware machine learning models includes updating a portion of the set of machine learning models. Each context-aware machine learning model of the set of context-aware machine learning models may be trained using a respective corresponding machine learning model of the set of machine learning models. Each respective context-aware machine learning model of the set of context-aware machine learning models may be smaller than the respective corresponding machine learning model such that a memory space and computation requirements of the respective context-aware machine learning model is less than half the memory space and the computation requirements of the respective corresponding machine learning model. The at least one processor may be configured to obtain copies of each context-aware machine learning model of the set of context-aware machine learning models for local execution on the one or more processors.

In some embodiments, analyzing the image data includes providing at least a portion of the image data to an artificial intelligence (AI) computer vision processing system including processing circuitry configured to perform the applying the at least the portion of the set of machine learning models to the image data. Each machine learning model of the set of machine learning models may be configured as a respective artificial neural network of a set of artificial neural networks. Each machine learning model of at least a portion of the set of machine learning models may be a convolution neural network (CNN) model. Each machine learning model of at least a portion of the set of machine learning models may be a network in network (NiN) model. Each machine learning model of at least a portion of the set of machine learning models may be a deep neural network (DNN) model.

In some embodiments, providing the at least the portion of the image data includes uploading, via a network connection, the image data to the AI computer vision processing system. The AI computer vision processing system may be executed in a cloud computing system. The AI computer vision processing system may be executed on an edge server co-located with the patient data capture apparatus. The patient data capture apparatus may include the edge server.

In one aspect, a medical procedure detection system for automatically identifying patient care at an emergency medical scene includes at least one camera, at least one light detection and ranging (LiDAR) sensor, at least one memory configured to store an electronic patient care record (ePCR), camera data, and LIDAR sensor data, and at least one processor communicatively coupled to the at least one camera and the at least one LiDAR sensor. The at least one processor may be configured to monitor the camera data and the LiDAR sensor data for evidence of initialization of a medical procedure provided to a patient by an EMS caregiver, identify the initialization of the medical procedure, responsive to the identification, store, to the memory, subsequent camera data and subsequent LiDAR sensor data, analyze the subsequent camera data and the subsequent LiDAR sensor data to identify performance of the medical procedure, and populate the ePCR based on the identified performance of the medical procedure.

In some embodiments, the initialization of the medical procedure includes at least one of a) preparation for providing the medical procedure or b) a first step in the performance of the medical procedure. Monitoring may include collecting, continuously in real-time in by repeatedly overwriting a temporary cache memory location, at least one of a camera data stream from the at least one camera, or a LiDAR sensor data stream from the at least one LiDAR sensor, and analyzing the at least one of the camera data stream or the LiDAR sensor data stream to recognize a precursor step to performance of a given at least one medical procedure of a set of medical procedures.

In some embodiments, the medical procedure detection system further includes a data cache communicatively coupled to the at least one camera and the at least one LiDAR sensor, where the data cache includes the temporary cache memory location. The memory may include the temporary cache memory location.

In some embodiments, the at least one LiDAR sensor is configured to provide the LiDAR sensor data to the at least one processor as point cloud data. The at least one LiDAR sensor may be configured to generate three-dimensional point cloud data. The medical procedure detection system may include a video image capture device, where the video image capture device includes a first camera of the at least one camera. The video image capture device may include a wearable video image capture device. The at least one camera may be configured to capture three-dimensional image data. The at least one camera may be configured as a handheld device, a wearable device, or a combination thereof. The wearable device may include a smart glasses device, a smart watch device, or a body camera.

In some embodiments, the at least one camera is configured to mount to or is integrated into another item of EMS equipment. The another item of EMS equipment may include a medical transport vehicle, medical equipment, a gurney, or a computer tablet.

In some embodiments, the at least one LiDAR sensor is configured as a handheld device, a wearable device, or a combination thereof. The wearable device may include a smart glasses device, a smart watch device, or a body camera.

In some embodiments, i) one or more cameras of the at least one camera and/or ii) one or more LiDAR sensors of the at least one LiDAR sensor are mounted to or integrated into a remotely controlled movable mount. The remotely controlled movable mount may be a drone. The remotely controlled movable mount may be configured to track a position of a patient or a caregiver.

In some embodiments, a first computing device includes the at least one processor and a second computing device includes the memory. Storing the subsequent camera data and the subsequent LiDAR sensor data may include transferring the subsequent camera data and the subsequent LiDAR sensor data, via a communication interface, to the second computing device. The first computing device may be an ePCR device. The second computing device may be an edge server. The communication interface may be a wireless interface.

In some embodiments, the at least one processor is further configured to, prior to transferring the subsequent camera data, converting the subsequent camera data to a smaller format. The smaller format may be a reduced resolution format. The smaller format may be a wireframe format.

In some embodiments, the at least one processor is further configured to archive a portion of the subsequent camera data and a portion of the subsequent LiDAR sensor data capturing the performance of the set of steps corresponding to the given medical procedure to an archive memory region. The portion of the subsequent camera data and the portion of the subsequent LiDAR sensor data may be archived to a second computing device. A cloud computing system may include the second computing device.

In some embodiments, analyzing the subsequent camera data and the subsequent LiDAR sensor data to identify performance of the set of steps includes applying one or more machine learning models of a set of machine learning models to at least one of the subsequent camera data or the subsequent LiDAR data. The medical procedure detection system may further include a cloud-based machine learning training architecture configured for execution on the cloud computing system, where the cloud-based machine learning training architecture is configured to apply the portion of the subsequent camera data and the portion of the subsequent LiDAR sensor data to updating the training of at least a portion of the set of machine learning models. The medical procedure detection system may include a cloud-based performance analysis architecture configured for execution on the cloud computing system, where the cloud-based performance analysis architecture is configured to analyze the portion of the subsequent camera data and the portion of the subsequent LiDAR sensor data to assess performance of at least one caregiver in performing one or more steps of the set of steps of the given medical procedure. The at least one processor may be further configured to, prior to archiving the portion of the subsequent camera data and the portion of the subsequent LiDAR sensor data, compressing the portion of the subsequent camera data and the portion of the subsequent LiDAR data.

The foregoing general description of the illustrative implementations and the following detailed description thereof are merely exemplary aspects of the teachings of this disclosure, and are not restrictive.

### BRIEF DESCRIPTION OF THE DRAWINGS

The accompanying drawings, which are incorporated in and constitute a part of the specification, illustrate one or more embodiments and, together with the description, explain these embodiments. The accompanying drawings have not necessarily been drawn to scale. Any values dimensions illustrated in the accompanying graphs and figures are for illustration purposes only and may or may not represent actual or preferred values or dimensions. Where applicable, some or all features may not be illustrated to assist in the description of underlying features. In the drawings:

FIG. 1A is a diagram of an example electronic patient care record user interface configured for manual data population;

FIG. 1B is an operational flow diagram of an example process for automatically identifying an emergency medical procedure using image data analysis and populating information regarding the procedure in an electronic patient care record;

FIG. 1C is an operational flow diagram of an example process for automatically identifying an emergency medical procedure using image data analysis and categorizing the procedure for invoicing purposes;

FIG. 1D is an operational flow diagram of an example process for automatically identifying an emergency medical procedure and assessing quality of procedure performance using image data analysis;

FIG. 2 illustrates image data capture of an example emergency medical scene using multiple image sensors positioned at multiple vantage points;

FIG. 3 is a block diagram of an example patient charting system;

FIG. 4A and FIG. 4B are a block diagrams of an example systems for automatically populating electronic patient charting data based on analysis of medical procedures captured in image data;

FIG. 5A through FIG. 5C are schematic illustrations of examples of emergency medical procedure identification and usage in the functioning of a patient data charting service;

FIG. 6 is a flow chart of an example method for analyzing image data to identify an emergency medical procedure;

FIG. 7A and FIG. 7B are swim lane diagrams of an example method for analyzing image data to identify an emergency medical procedure and automatically evaluate the performance of the procedure;

FIG. 8A through FIG. 8C are block flow diagrams of example processes for training machine learning models to analyze image data to identify emergency medical procedures;

FIG. 8D is a block diagram of an example process for training medical equipment recognition models for identifying medical equipment items used in each of a collection of medical procedures; and

FIG. 9 illustrates an example of a logical and physical architecture of a patient charting service as part of an SaaS platform.

### DETAILED DESCRIPTION OF ILLUSTRATIVE EMBODIMENTS

To alleviate or eliminate some of the difficulties associated with encounter documentation in the pre-hospital environment, rescuers can benefit from tools that provide automatic recordation. Such tools may collect, integrate, analyze, and record information for a patient encounter. Rescuers can also benefit from tools that provide guidance for care.

Often in an emergency encounter, an EMS caregiver interacts with a critically ill patient under circumstances that require the most efficient intervention possible. The emergency encounter is often in a non-medical environment like a home, office, or gym. In many cases, the encounter occurs in the chaotic environment of a fire scene, a car accident, or a mass casualty scene.

In addition to challenging environments, the EMS caregiver is tasked not only with helping patients but also with recording information descriptive of the encounter and the patient. The EMS caregiver, for example, must typically document clinical observations of the patient, demographic information, therapies and interventions provided to the patient, timing of therapy and interventions, causes of injury or illness, medical history, etc. Such documentation may be needed, for example, for protocol adherence, for care guidance, for medical records, and/or for medical billing purposes.

The ePCR may include multiple data set sections that cover various aspects of the documentation of an encounter between EMS crew and a patient. The encounter may be an emergency encounter or a non-emergency encounter such as a pre-scheduled transport to a medical facility or between medical facilities, community paramedicine, etc. The data set section may include, for example, data sets for airway, cardiac arrest, EMS crew, medical device, dispatch, patient disposition, patient examination, patient history, injury, laboratory results, and medications. There may also be custom configurations and sections. As an example, a patient history section may include the data fields indicated below in Table 1. Examples of field values for the data fields are also provided in Table 1. The data field values may be associated with an International Classification of Diseases (ICD) code or other medical code for billing purposes.

TABLE 1

| Data field | Field value |
|---|---|
| Last name of Patient's Practitioner | Smith |
| First name of Patient's Practitioner | Chris |
| Advance Directives | none |
| Medication Allergies | Penicillin |
| Environmental/Food Allergies | Peanut |
| Medical/Surgical History | Type 2 diabetes |
| Medical History Obtained From | Patient's husband |
| Patient's Immunization | Flu |
| Immunization Year | Current year |
| Current Medications | Metformin |
| Current Medication Dose | 500 |
| Current Medication Dosage Unit | mg |
| Current Medication Administration | Oral |
| Alcohol/Drug Use | none |
| Pregnancy | yes |
| Last Oral Intake | none |

As another example of ePCR data, Table 2 below shows examples of data fields and data field values for ePCR documentation of a pre-scheduled dialysis transport.

TABLE 2

| Data field | Field value |
|---|---|
| Call Source | Phone call |
| Dispatch Center | Verifast EMS Services |
| Run Number | 47 |
| Incident Number | 56-87 |
| Dispatched Complaint | Palliative Care |
| Patient Acuity at Dispatch | Priority 4 (Non-Acute) |
| Changed Priority | Pre-Scheduled |
| Trauma call type | Medical and trauma |
| Call type | BLS |
| Response Mode | Pre-Scheduled |
| Additional Response Mode | No lights or Sirens |
| Pickup Zone | 16 |
| Response Delay | None |
| Type of Service | Interfacility transport |
| Patient Disposition | Treated & transported |

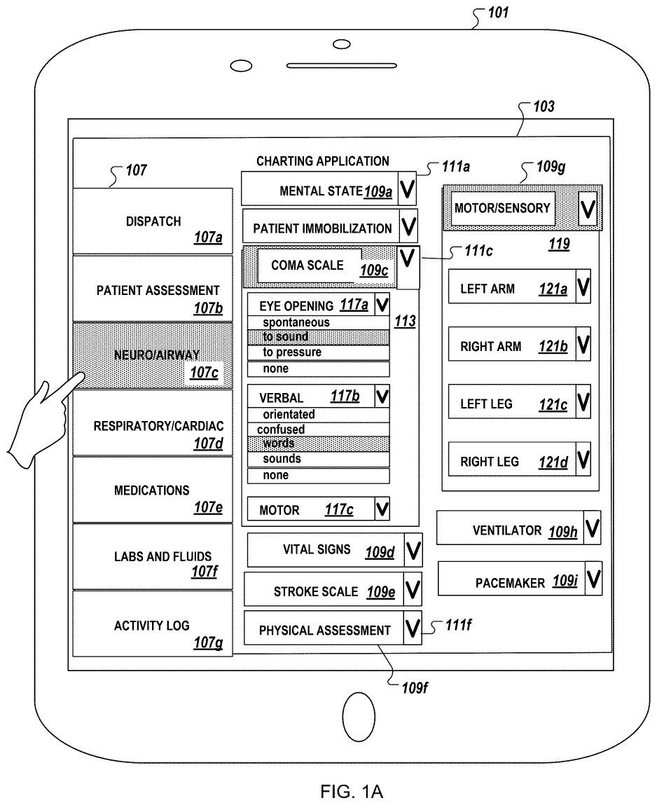

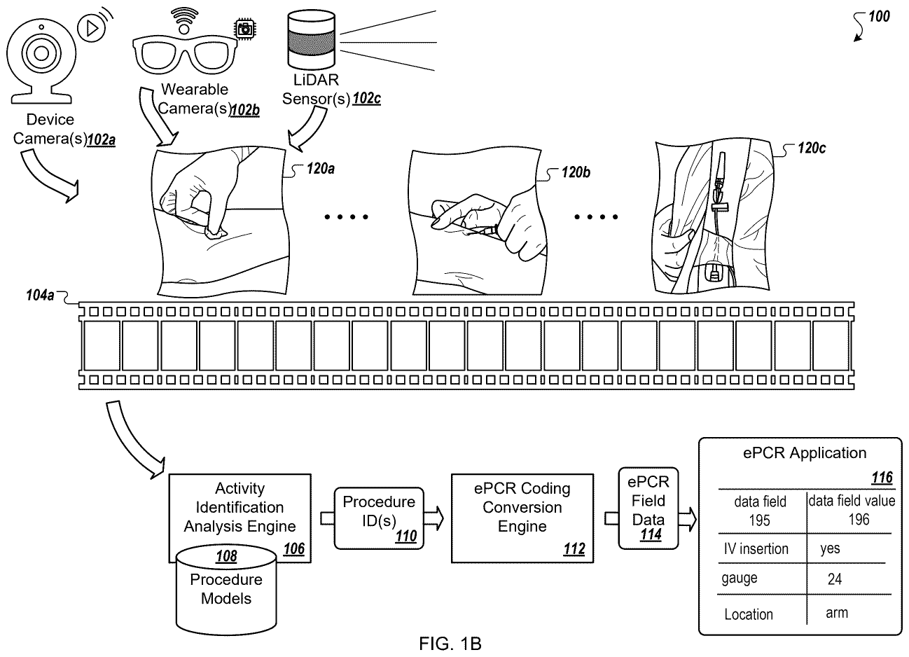

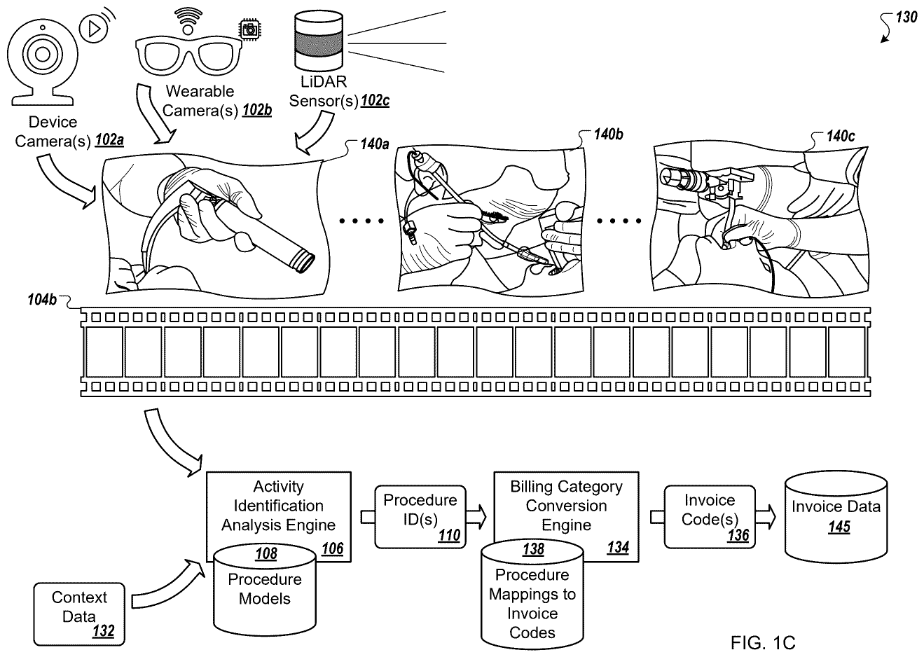

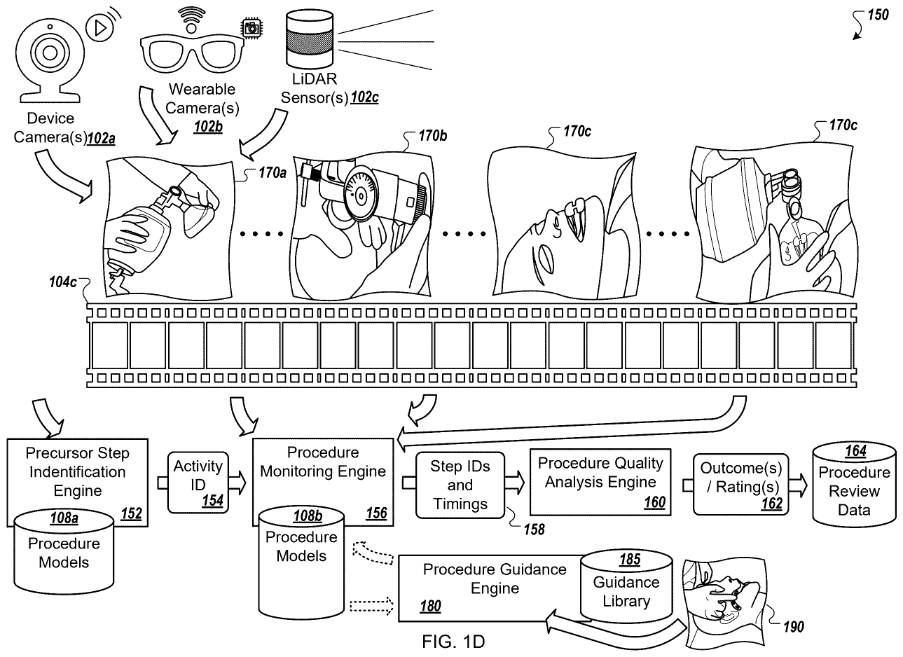

Referring to FIG. 1A, an example of a manually populated ePCR is shown. A computing device 101 may provide a user interface 103 for a charting application. For a complete ePCR, the charting application may allow the user to walk through the multiple pages 107a-g of the ePCR. A selection of a page 107 (e.g., a neuro/airway page 107c) may display multiple data categories 109, each with a drop-down menu control 111 or another control that allows a display of the information needed for the data category. For example, the selection of “coma scale” category 109c using drop down menu control 111c exposes a menu 113 with subcategories for coma scale. Each category and/or subcategory may correspond to one or more data fields that each require entry of field value. For example, the category of coma scale corresponds to the data fields 117 of “eye opening” 117a, “verbal” 117b, and “motor” 117c. The data field “eye opening” 117a requires a field value of “spontaneous,” “to sound,” “to pressure,” or “none” with “to sound” selected in this example. Similarly, the data field of “verbal” 117b requires a field value of “orientated,” “confused,” “words,” “sounds,” or “none” with “words” selected in this example. The selection of a “motor/sensory” category 109g exposes a menu 119 with subcategories 121 for motor/sensory. Each of the subcategories “left arm” 121a, “right arm” 121b, “left leg” 121c, and “right leg” 121d is a data field that requires entry of a data field value. The Tables 1 and 2 show above show examples of data fields and field values specific to the particular categories of patient history and dialysis transport. In order to complete the ePCR, the user may need to step through all or most of the categories and subcategories and enter data field values for all of the data fields in these categories and subcategories. The total number of required data field values may be on the order of 50-1000 as discussed above.

In light of these issues, automated ePCR data capture may provide accurate and hands-free contemporaneous ePCR data capture during the patient encounter without the reductions in data accuracy and efficacy of care as discussed above. Unlike data entered after completion of a patient encounter, the contemporaneous data entry reduces or eliminates data entry errors and/or the amount of missing required information (e.g., based on a data entry standard such as NEMSIS) for a particular call type or protocol. Furthermore, such a system eliminates the need for caregivers to divert time and attention away from patient care for the purpose of documentation. For example, medics can use their hands to take a pulse, inject drugs, and apply cardiopulmonary resuscitation (CPR) rather than take notes on a glove or hold and enter data into a computer tablet. Automated data capture minimizes, and in some instances, eliminates manual human interference in data capture for the ePCR.

As discussed above, to provide a complete and accurate record of each encounter with a patient, including patient information and treatment/intervention information, an EMS caregiver may complete the electronic patient care record (ePCR). ePCRs include data fields configured to store a comprehensive set of patient and encounter information according to a schema that controls the structure of the data provided to the digital record. In some implementations, the schema is embodied by a multi-agency standard that provides a compliance architecture to allow transfer of data and data interoperability between individual agency systems and to enable entry of data in a centralized database. An example of such a standard for documentation of patient care information is the National Emergency Medical Services Information Standard (NEMSIS) for emergency care medical record data collection. In some implementations, the standard for documentation may be a regional standard that may be similar to NEMSIS but affords a local jurisdiction the flexibility to tailor the documentation based on local agency and governmental preferences. For example, the state of New Hampshire uses a web-based statewide data system, the Trauma Emergency Medical Services Information System (TEMSIS). TEMSIS enables EMS agencies in New Hampshire to collect their own data and then upload to the state via XML or to enter run or encounter data via a web browser using an online form.

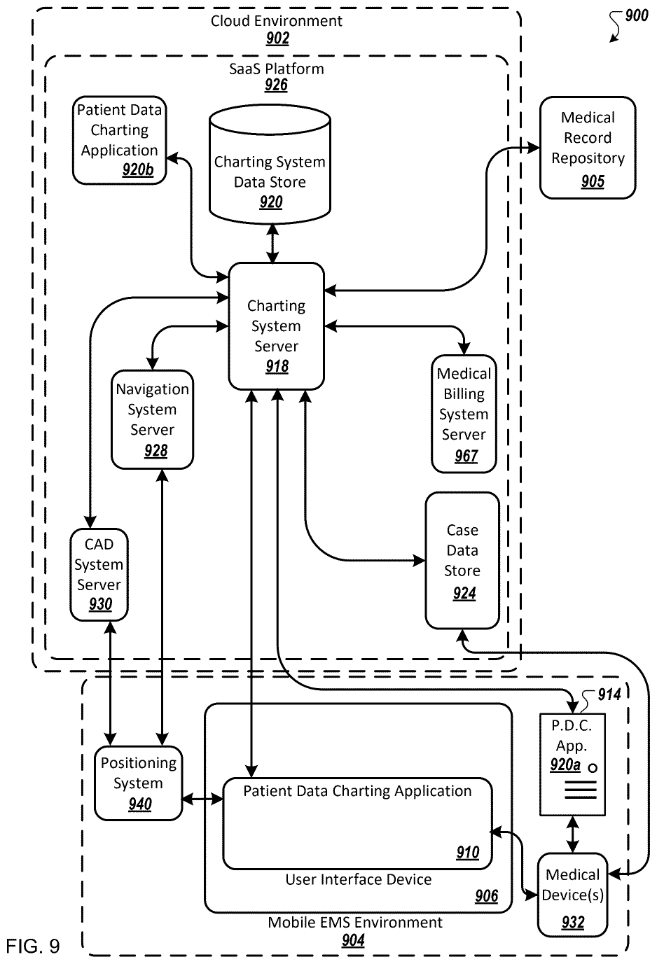

In order to transfer data between disparate healthcare systems (e.g., between the systems serving an EMS agency and those serving a hospital, between different EMS agencies, between different hospitals, between medical providers and medical payors, etc.), an ePCR system (e.g., the charting system server 918, the charting system data store 920, and the patient data charting application 920b as shown in FIG. 9) may utilize a communication messaging standard such as, for example, a Health Level Seven (HL7®) version 2, HL7® version 3, or a Health Level Seven Fast Healthcare Interoperability Resources (HL7® FHIR®) standard. These messaging standards may also enable communications with a medical billing system server 967 and/or medical record repository 905, as shown in FIG. 9. Data may also be stored in an HL7® or HL7® FHIR® messaging format. Other formats and/or communication standards may include, for example, Clinical Document Architecture (CDA) standard, an Electronic Data Interchange (EDI) Healthcare standard (including, e.g., 270, 271, 276, 277, 278, 820, 834, 835, 837P, and/or 8371 standard). CDA and EDI may be associated with hospital medical records and may not be associated with an ePCR generated by an EMS agency. Procedures and codes used to describe and categorize medical conditions within an electronic medical record may conform to a standardized nomenclature or code such as, for example, the Systematized Nomenclature of Medicine Clinical Terms (SNOMED CT®), International Classification of Diseases (ICD), Healthcare Common Procedure Coding System (HCPCS), and/or Current Procedural Terminology (CPT®) coding standard.

In some situations, ePCRs may be only partially completed during an encounter, or require a dedicated documentarian, because the attention and focus of the EMS caregiver may be necessarily and properly with the patient. Post encounter completion of the ePCR may increase inaccuracies and may introduce delays into the overall continuity of care provided to the patient. In some situations, for example where the urgent needs of a patient render documentation difficult or impractical, EMS caregivers may resort to recordation short-cuts, such as writing notes on scrap paper, backs of gloves, ECG tape, or other readily available handwriting stock. This is particularly true if the documentation process relies on hands-on data entry. For example, data entry to a computing device, such as a tablet computer, laptop computer, or other mobile device processing the ePCR may require manual entry via a touchscreen, keyboard, stylus, or other manual data entry device. This aspect of ePCR screens can make it time-consuming and difficult to enter patient and encounter information.

As another factor, in some implementations, the ePCR may include 50-1000 fields for which a data entry is required (e.g., required by laws of a state or another jurisdiction and/or required for adherence to a data collection standard, etc.). Since the user may not be able to reduce or customize the number of data entry fields, at least at the point of care, the accuracy and completeness of the ePCR may improve as a result of automated filling of at least a portion of these fields. This may reduce or eliminate inaccurate and/or incomplete data entry which can detrimentally affect patient care, patient outcomes, and the quality and usefulness of information passed from an emergency care encounter to a subsequent hospital encounter.

NEMSIS is just one example of an official EMS data collection standard for EMS agencies which allows transfer of data between systems and provides a national EMS repository for reporting and research. NEMSIS provides consistent data field requirements and definitions for electronic records generated by EMS for emergency care and non-emergency out-of-hospital care (e.g., scheduled transports, community paramedicine, etc.). The NEMSIS data collection via NEMSIS-compliant ePCRs may enable analysis of this data for evaluation of and evidence-based improvements in patient care across an array of EMS agencies. In particular, the NEMSIS-compliant ePCRs conform to a structured XML standard for the ePCR data. The NEMSIS standard is an example only and other formats and/or content requirements are within the scope of this disclosure.

Thus, in accordance with at least some examples disclosed herein, a patient data capture apparatus is described for automatically populating ePCR data at an emergency medical scene by capturing and analyzing image data to identify emergency medical procedures performed on a patient by caregivers. The image data, for example, may be collected by one or more devices as still and/or video image data. The devices, for example, may include medical equipment at an emergency medical scene having one or more image sensors, image sensors worn by caregivers, dedicated image capture devices, and/or portable computing devices with image sensors (e.g., a tablet computer or laptop computer held by a caregiver or disposed at the emergency medical scene in a manner in which the image sensor(s) capture procedures performed on the patient). Further, in addition to and/or rather than visible data (e.g., taken by a camera device), the image data may include, in some examples, heat map data and/or LiDAR data. Analyzing the image data may include identifying a sequence of steps corresponding to a particular medical procedure. One or more machine learning models, for example, may be trained to recognize medical equipment used in certain medical procedures as well as locations on a patient's body where the medical equipment is used (e.g., inserted, applied, or tethered). Upon recognizing a medical procedure, at least one ePCR entry may be automatically recorded to log performance of the medical procedure. In this manner, the patient charting apparatus provides the benefit of documenting procedures in real-time or near real-time as the procedures are performed without competing with caregiver attention to the patient. In some embodiments, a caregiver may be presented information regarding the recognized medical procedures so that the caregiver can confirm accuracy of the recognition of the medical procedure. This confirmation, for example, may reduce and streamline the documentation burden as compared with entry of an entire procedure.

The patient data capture apparatus is configured to recognize certain medical procedures, in some embodiments, based in part on contextual information regarding the emergency medical scene. The contextual information, in one example, may include context data from the medical equipment used, such as alarms, alerts, and/or data metrics collected by the patient data charting apparatus from the medical equipment and/or recognized via image data of the medical equipment (e.g., display readings, illuminated error lamps, etc.). For example, the contextual data may include physiological metrics gathered by the medical equipment. In another example, the contextual information may include context data regarding the patient and/or nature of the medical emergency, as already populated in ePCR data records (e.g., a type of emergency medical vehicle and/or crew deployed, medical dispatch codes applied when dispatching the caregivers to the medical emergency, a skill level or certification level of caregiver(s) involved in the medical procedure being performed, etc.). The contextual information, for example, may be used to select between two similar medical procedures. In particular, where a portion of the steps of the medical procedure may be missing from the image data due to the image sensor(s) having a blocked vantage point, the context may be used to increase confidence in the identification of the emergency medical procedure. In a further example, contextual data may be used in machine learning implementations to reduce the size of machine learning models used, allowing for real-time analysis performed using portable field equipment lacking the increased processing and memory capabilities commonly allocated to machine recognition tasks.

In accordance with at least some examples disclosed herein, a patient data capture apparatus is configured to identify patient care at an emergency medical scene to identify a medical procedure and select an appropriate billing category corresponding to the medical procedure. Rather than relying on a caregiver to manually search and enter appropriate billing codes and/or rather than relying on an automated system to provide billing codes to a record after the medical and demographic data has already been entered, the systems and methods described herein may enable a recognition and entry of medical codes at approximately the same time that the medical information is recorded in the ePCR. For example, the systems described herein may recognize and identify a type of equipment and/or a type of caregiver through analyzing image data captured at the emergency medical scene and automatically identify the billing category (e.g., basic life support (BLS), advanced life support (ALS), ALS level 1 (ALS1), and ALS level 2 (ALS2)) and/or billing codes approximately concurrently with the recognition and identification.

In accordance with at least some examples disclosed herein, a patient data capture apparatus is configured to evaluate a caregiver's performance of the medical procedure and log performance metrics regarding the performance. The patient data capture apparatus may be configured to recognize individual caregivers at the emergency medical scene and associate the performance metrics with a particular caregiver performing the medical procedure. In one example, the patient data capture apparatus may be configured to perform facial recognition of caregivers on scene to identify a particular caregiver. In another example, the patient data capture apparatus may be configured to perform natural language processing and/or analyze a machine-readable code printed on a caregiver badge to identify the particular caregiver. Evaluating the caregiver's performance may include evaluating a time to perform certain steps of a medical procedure, evaluating a number of repetitions of certain steps of the medical procedure, and/or evaluating a success or failure of completion of the medical procedure. Quality metrics derived by the patient data capture apparatus through evaluating performance, for example, may be stored in association with the caregiver, thereby providing the benefit of building information regarding caregivers with the most experience in certain medical procedures, caregivers most efficient and/or proficient in certain medical procedures, and/or caregivers who require more training and/or exposure to certain medical procedures.

The description set forth below in connection with the appended drawings is intended to be a description of various, illustrative embodiments of the disclosed subject matter. Specific features and functionalities are described in connection with each illustrative embodiment; however, it will be apparent to those skilled in the art that the disclosed embodiments may be practiced without each of those specific features and functionalities.

Reference throughout the specification to “one embodiment” or “an embodiment” means that a particular feature, structure, or characteristic described in connection with an embodiment is included in at least one embodiment of the subject matter disclosed. Thus, the appearance of the phrases “in one embodiment” or “in an embodiment” in various places throughout the specification is not necessarily referring to the same embodiment. Further, the particular features, structures or characteristics may be combined in any suitable manner in one or more embodiments. Further, it is intended that embodiments of the disclosed subject matter cover modifications and variations thereof.

It must be noted that, as used in the specification and the appended claims, the singular forms “a,” “an,” and “the” include plural referents unless the context expressly dictates otherwise. That is, unless expressly specified otherwise, as used herein the words “a,” “an,” “the,” and the like carry the meaning of “one or more.” Additionally, it is to be understood that terms such as “left,” “right,” “top,” “bottom,” “front,” “rear,” “side,” “height,” “length,” “width,” “upper,” “lower,” “interior,” “exterior,” “inner,” “outer,” and the like that may be used herein merely describe points of reference and do not necessarily limit embodiments of the present disclosure to any particular orientation or configuration. Furthermore, terms such as “first,” “second,” “third,” etc., merely identify one of a number of portions, components, steps, operations, functions, and/or points of reference as disclosed herein, and likewise do not necessarily limit embodiments of the present disclosure to any particular configuration or orientation.

Furthermore, the terms “approximately,” “about,” “proximate,” “minor variation,” and similar terms generally refer to ranges that include the identified value within a margin of 20%, 10% or preferably 5% in certain embodiments, and any values therebetween.

All of the functionalities described in connection with one embodiment are intended to be applicable to the additional embodiments described below except where expressly stated or where the feature or function is incompatible with the additional embodiments. For example, where a given feature or function is expressly described in connection with one embodiment but not expressly mentioned in connection with an alternative embodiment, it should be understood that the inventors intend that that feature or function may be deployed, utilized or implemented in connection with the alternative embodiment unless the feature or function is incompatible with the alternative embodiment.

FIG. 1B through FIG. 1D illustrate example processes including automatically identifying an emergency medical procedure using image data 104 collected by one or more image devices 102. As illustrated in FIG. 1B through FIG. 1D, the image data 104 may be collected by a variety of image devices 102, such as one or more computing device and/or medical device cameras 102a, one or more wearable cameras 102b, and/or one or more LiDAR sensors 102c. The one or more device cameras 102a, in some examples, may be included in a tablet computer, smart phone, laptop computer patient monitoring device, patient monitoring and therapy device, and/or a patient therapy device. For instance, a patient data charting device (e.g., ePCR device) may include a built-in camera 102a. Further, the one or more device cameras 102a may include one or more standalone camera devices, such as an action camera (e.g., a GoPro camera by GoPro Inc. of San Mateo, CA) or a computer peripheral camera device (e.g., a webcam). The one or more wearable cameras 102b, in some examples, can include a smart glasses device, a smart watch device, or a body camera. The device cameras 102a and/or the wearable cameras 102b may each be configured to capture video and/or still images. Further, certain device cameras 102a and/or wearable cameras 102b may be configured to collect thermal images (e.g., Forward Looking Infrared (FLIR) images by Teledyne FLIR LLC of Wilsonville, Oregon). The one or more LiDAR sensors 102c may collect wireframe data and/or point cloud data. The LiDAR sensor(s) 102c may be configured to collect two-dimensional and/or three-dimensional LiDAR sensor data. Further, one or more of the image devices 102 may be computer vision cameras including object recognition algorithms, artificial intelligence, and/or edge computing processing capabilities.

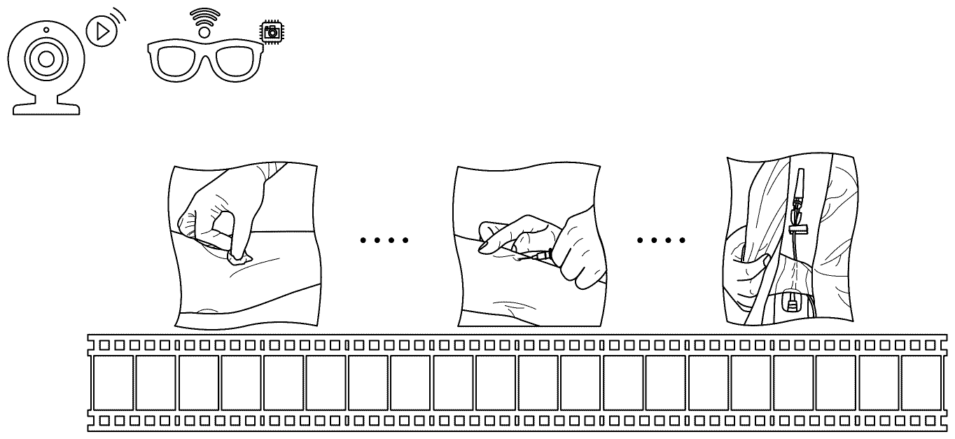

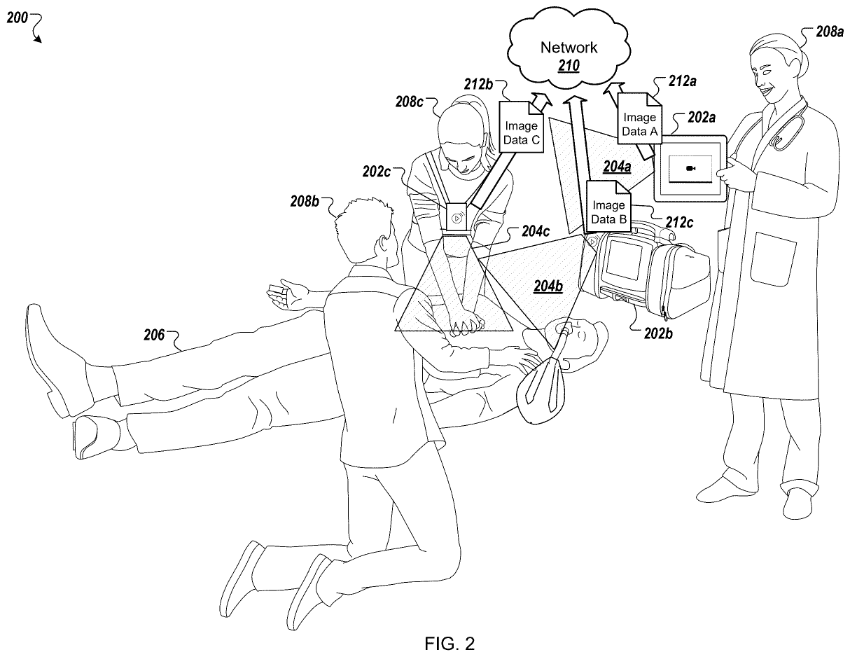

An example arrangement of image devices deployed at an emergency medical scene for capturing image data of medical procedures is illustrated in FIG. 2. Turning to FIG. 2, in some implementations, image data 212a-c is captured at an emergency medical scene 200 by a set of image devices 202 including a tablet device 202a held by a supervising caregiver 208a, a patient data collection and monitoring device 202b (e.g., a defibrillation device) disposed near a supine patient 206, and a bodycam device 202c worn by a chest compression-providing caregiver 208c. As illustrated, each image device 202a-c captures image data 212a-c from a different vantage point 204a-c. In this manner, if the compression-providing caregiver 208c or a respiration-providing caregiver 208b moves to a position blocking, for example, the vantage point 204b of the patient data collection and monitoring device 202b, the image data 212a of the tablet 202a and/or the image data 212b of the bodycam 202c may still capture the emergency medical procedure. For example, the caregivers 208a-c may recognize that the patient 206 requires defibrillation, and at least a portion of the image data 212a-c may capture the defibrillation emergency medical procedure.

In some embodiments, the image data 212a-c captured by each image device 202a-c transferred via a network 210 for analysis (e.g., by a separate computing device). Although image data 212a-c from each image device 202a-c is illustrated as transmitting via the network 210, in other embodiments, the image data 212b-c from the image devices 202b and 202c may be transferred to the tablet 202a for analysis. The network 210, in some examples, may include wired and/or wireless connections to the separate computing device. In some embodiments, the network 210 is a local area network (LAN) or private area network (PAN) established between devices at the emergency medical scene 200. Data communications architectures for transferring image data are discussed in further detail below in relation to FIG. 9.

Returning to FIG. 1B through FIG. 1D, the image data 104, in some embodiments, is collected by the patient data capture apparatus for analysis. For example, an ePCR data capture device may be configured to collect the image data 104 from one or more image devices 102. The image data 104, in some examples, can include black and white still and/or video images, full color still and/or video images, full resolution (e.g., bitmap) images, and/or reduced resolution (e.g., compressed) images. In some implementations, one or more image data streams are combined (e.g., co-registered) in the image data 104. The image data 104 may also include wireframe and/or point cloud data (e.g., from the LiDAR sensor(s) 102c).

In some embodiments, analyzing the image data 104 includes identifying steps of a medical procedure. The medical procedure, in some examples, can include oxygen delivery, intravenous saline delivery, intravenous drug delivery, obtaining a set of vital physiological measurements, cricothyrotomy, nasal intubation, endotracheal intubation, and/or tourniquet application with infusion. Further, the medical procedure may be identified in part based on identifying one or more medical equipment items. The medical equipment, in some examples, can include gloves, a 3-lead EKG, a 12-lead EKG, a cardiac monitor, a cardioverter, a central intravenous (IV) catheter, an IV bag, a defibrillator, tubing, a ventilator, a bag valve mask, a tourniquet, a splint, a backboard, a cervical collar, a gurney, gauze, an alcohol swab, and/or a nasal cannula.

Turning to FIG. 1B, an operational flow diagram of an example process 100 for automatically identifying an emergency medical procedure using image data analysis and populating information regarding the procedure in an electronic patient care record is illustrated. As shown in FIG. 1B, a stream or set of image data 104a is provided to an activity identification engine 106 to identify a medical procedure 120 captured in the image data 104a. The medical procedure 120, as illustrated, is an intravenous (IV) needle insertion procedure.

In some embodiments, the activity identification engine 106 applies a collection of procedure models 108 to analyzing the series of steps 120a-c of the medical procedure 120, including an initial (e.g., precursor) step 120a of cleaning a spot on an interior of an elbow of the patient, an insertion step 120b of inserting the IV needle, and a conclusory step 120c of taping the IV needle into place. Further, the activity identification engine 106 may analyze the image data 104a in view of subsequent activity (e.g., maintenance of the IV needle in position, etc.) to determine completion and/or failure of the medical procedure 120. Although only three steps 120a-c (e.g., three images) of the IV needle insertion procedure are illustrated in the example of FIG. 1B, the process 100 may be configured to identify additional steps in recognizing the medical procedure 120 as an IV needle insertion procedure, such as obtaining and unwrapping the IV needle and/or palpitating the skin to find the vein.

The procedure models 108, in some implementations, are machine learning models trained to identify a collection of medical procedures including the intubation procedure. For example, each machine learning model 108 may be trained to identify a different medical procedure of a collection of medical procedures. Further, certain models of the collection of procedure models 108 may be configured to identify a particular medical procedure using a particular type or set of data (e.g., a model trained with color video data, a model trained with wireframe LiDAR data, a model trained with black & white image data along with medical equipment contextual data, etc.). The procedure models 108 may be part of a computer vision processing system. Some models of the collection of procedure models 108 may have an artificial neural network architecture, such as a deep neural network (DNN). Some models of the collection of procedure models 108 may have a convolution neural network (CNN) architecture. Some models of the collection of procedure models 108 may have a network-in-network (NiN) architecture.

In some implementations, the activity identification engine 106 is configured to identify, from analyzing the image data 104a with at least a portion of the procedure models 108, a procedure identifier 110. Further, in some embodiments, the activity identification engine 106 may provide a confidence rating (e.g., level, percentage accuracy, etc.) representing a likelihood of the procedure identifier 110 correctly identifying the medical procedure 120.

In some implementations, an ePCR coding conversion engine 112 translates the procedure identifier 110 into one or more codes for entering as ePCR field data 114. The ePCR data, in some examples, can be translated into a number of different PCR coding standards, such as SNOMED CT, HCPCS, CPT, ICD, etc. For example, the ePCR coding conversion engine 112 may be configured to interoperate with a number of different ePCR coding standards and/or standards versions. The ePCR coding conversion engine 112 may identify a first code related to the procedure, for example, and further identify, based on the field corresponding to the first code, one or more related data fields that are each procedurally related to the field. For example, an ePCR data structure may include logical associations between standard element identifiers signifying procedural associations. By following the logical associations (e.g., database links or data tags, etc.), the ePCR coding conversion engine 112 may map the procedure identifier(s) 110 to ePCR field data 114. For example, to successfully perform a certain medical procedure, a caregiver may need to use particular disposable medical equipment, connect the patient to a certain therapy device, and/or conduct particular interim steps of the procedure (e.g., according to a treatment protocol) which utilize certain medical equipment items, each of which may have a designated ePCR field for population. The ePCR coding conversion engine 112, for example, may access a defined intervention sequence of activities corresponding to the treatment protocol to identify the associated fields and/or the appropriate codes for populating the associated fields. If the values of certain fields are unknown (e.g., not recognized during analysis of the image data 104a), context data and/or caregiver prompts may be used to fill in the missing information. Context data is described in greater detail below, for example in relation to FIG. 1C.

In some implementations, a predictive workflow, for example a workflow derived through machine learning analysis of the medical procedures, is used to identify the ePCR data fields (e.g., sub-procedures, medical equipment items, etc.) associated with each medical procedure. A deep neural network predictive model 108, during training, may be used to obtain information regarding why certain series of steps are deemed to correspond to the trained medical procedure, and the “why” information may automatically populate a predictive workflow for use in managing ePCR field data identification by the ePCR coding conversion engine 112. Further, the predictive workflow may identify ePCR field selection factors (e.g., branches or selections between possible fields) and/or ePCR data field values that can be gleaned by the ePCR coding conversion engine 112 from context data such as, in some examples, a geolocation of the emergency transport vehicle (e.g., on scene, en route, arrived at hospital, etc.), a type of EMS service dispatched (e.g., ALS1, ALS2, etc.), and/or dispatch code(s) related to the dispatch of the emergency caregiver team.

In some implementations, the ePCR field data 114 is entered into a patient charting application 116. As shown, for example, in FIG. 1B, the ePCR application 116 may include data fields 195 and data field values 196 may be provided for each data field. The data fields and allowed data field values may be determined by the NEMSIS standard. In the example of FIG. 1B, the ePCR application may use the ePCR field data 114 to identify a corresponding data field 195 and then enter a data field value 196 into that data field based on the automated recognition of procedures as described herein. The ePCR field data 114, for example, may identify the medical procedure 120 as well as, in some embodiments, contextual data such as type of equipment used (e.g., type of tubing), type of disposable medical items used (e.g., such as the gloves used in steps 120a-c), and a time at which intubation was begun and/or completed. In some embodiments, the ePCR fields are organized according to medical procedure categories, such as a respiratory section and a cardiac section, and the ePCR field data 114 identifies a category and medical procedure. The patient charting application 116, in turn, may present to a caregiver (e.g., visually and/or audibly) a confirmation of submission of information related to the procedure identified by the procedure identifier 110.

In some implementations, instead of and/or in addition to recording ePCR field data 114, the procedure identifier 110 may be used in identifying a billing category for use with invoicing. Turning to FIG. 1C, in some implementations, an example process 130 for automatically identifying an emergency medical procedure using image data analysis and categorizing the procedure for invoicing purposes is illustrated. The process 130 includes the activity identification engine 106 and procedure models 108, configured to identify procedure identifiers 110. However, in the process 130, the activity identification engine 106 further accesses context data 132.

The medical procedure 140, as illustrated, is an intubation procedure. Although only three steps 140a-c (e.g., three images) of the intubation procedure are illustrated in the example of FIG. 1C, the process 130 may be configured to identify additional steps in recognizing the medical procedure 140 as an intubation procedure.

In some embodiments, the activity identification engine 106 accesses the context data 132. The context data 132, can include, in some examples, patient medical information and/or patient demographic information. In some implementations, at least a portion of the context data 132 is derived through analyzing the image data 104b and/or other previous and/or concurrently captured image data of the emergency medical scene. The patient medical information may include, in non-limiting illustration, an identifier of a medication from a medication label, electrocardiogram (ECG) information (e.g., recognized from an ECG tape and/or from a screen shot of a medical device display), or patient physiological information recognized from a screen shot of a medical device display. In a further example, patient physiological information may be recognized through analyzing image data of the patient, such as infrared image data to determine patient temperature, pulse rate, and/or respiration rate, or video image data to evaluate patient breathing metrics (e.g., rate, pause between, consistency of breathing pace, evidence of hyperventilation, etc.) based on chest movements. The patient demographic information, in some examples, may be derived through natural language processing (NLP) of driver's license information, insurance card information, and/or patient information from a face sheet. The NLP analysis may include recognizing handwritten text. The image data analyzed to identify the context data 132 may differ than the image data analyzed to recognize medical procedures. In an illustration including multiple image capture devices, an image capture device directed toward a patient monitoring device display may be used to derive context data 132, while another one or more image capture devices directed toward a patient may be used to obtain the image data 104b for recognizing medical procedures.

In some embodiments, at least a portion of the context data 132 is collected from non-image data sources. For example, rather than or in addition to evaluating image data to recognize patient medical information, patient physiological data, including physiological metrics (e.g., heart rate, respiration rate, and/or blood oxygen level, etc.) or other physiological data (e.g., ECG sensor data, EKG sensor data, heart sound monitoring sensor data, etc.), may be provided by one or more medical devices on scene for inclusion in the context data 132. The physiological data may further include device alert or alarm data from one or more medical devices, such as, in some examples, a ventilator alarm, a bag valve mask sensor alarm, an impedance sensor alarm, and/or an airflow sensor alarm. Alarms may also include one or more of breathing circuit leak alarm, high or low end tidal CO2 alarm, low saturated oxygen (SpO2) alarm, low fraction of inspired oxygen (FiO2) alarm, high or low volume, high or low pressure, high or low breath rate, etc. Further, patient demographic information may be gathered into context data 132 from ePCR data or other patient information data provided to the caregivers (e.g., via a dispatching program, etc.). The demographics, in some examples, can include age and/or gender.

In some embodiments, the context data 132 includes caregiver information. The caregiver information, for example, may be obtained through analyzing the image data 104b or other prior and/or concurrent image data. For instance, a caregiver badge may be used to identify a name, a certification level, an employee level, or a skill level of the caregiver through natural language processing and/or reading a machine-readable indicia (e.g., barcode, quick-response (QR) code, etc.). In another illustration, facial recognition may be performed on image data to recognize a particular caregiver out of a collection of professionals. Further, the name, face, and/or other information derived through image analysis may be used to obtain information regarding the caregiver, such as the certification level, employee level, and/or skill level (e.g., by querying a database of professionals). In another illustration, caregiver information may be derived from a separate data source, such as team member information supplied in a dispatch order. The employee level of a caregiver, in some examples, can include an emergency medical technician, a paramedic, a medic, a physician, a nurse, and/or a medical scribe. Certification levels, in some examples, can include a basic life support (BLS) certification, a community paramedic certification (CP-C), a critical care paramedic certification (CCP), a certified nurse assistant certification (CAN), a medical assistant certification, and/or an advanced emergency medical technician (AEMT). Skill levels, in some examples, can include levels of training of caregivers, levels of experience (e.g., novice, knowledgeable, expert, etc.), and/or levels of autonomy (e.g., trainee, team member, supervisor, trainer, etc.) with respect to a given medical procedure.

The context data 132, in some embodiments, includes identification of medical equipment used prior to and/or during performance of the medical procedure. The medical equipment may include, in some examples, first aid equipment (e.g., alcohol swabs, tubing, gloves, gauze, a central intravenous (IV) catheter, an IV bag, a splint, a tourniquet, a backboard, a cervical collar, a gurney, and/or a nasal cannula, etc.), patient monitoring equipment (e.g., a 3-lead EKG, a 12-lead EKG, a cardiac monitor, etc.), and/or patient therapy equipment (e.g., a cardioverter, a defibrillator, a ventilator, a bag valve mask, and/or an automated chest compression device, etc.). In some implementations, a portion of the medical equipment is identified through analyzing image data, such as the image data 104b, to recognize identification markings on the equipment. The identification markings, for example, can include a color and/or shape assigned to each type of medical equipment item. In illustration, the cardiac monitor may be identified through application of a red heart sticker, while the defibrillator is identified using a yellow lightning bolt sticker. In some embodiments, certain medical equipment items are identified through words and/or codes applied to the equipment. The words and/or codes may be added to the equipment and/or included during the manufacturing process, such as a product name printed on the front of a ventilator.

In some implementations, a portion of the medical equipment is identified through communications or other signals provided by the individual pieces of medical equipment. For example, an energy signature of a particular medical device may be recognized and stored as context data (e.g., the energy signature of a wearable quality analysis and/or caregiver prompting device, a therapy device, or a patient monitoring device). In another example, proximity data derived from near-field wireless communications or short-range wireless communications may provide the identity and/or proximity of the medical device. The proximity, for example, may be indicative of the equipment being in use (e.g., being moved closer to the patient, even if the equipment is not visible to the one or more image capture devices). Communication signals or signatures, further, may be used to identify a particular device (e.g., through device identifier or other data individually identifying a piece of equipment and/or the type of that equipment).

In some embodiments, the activity identification engine 106 applies the collection of procedure models 108 to analyzing the series of steps 140a-c of the medical procedure 140 in view of the context data 132, including an initial (e.g., precursor) step 140a of accessing the tubing, an insertion step 140b of directing the tubing into the mouth and down the throat of the patient, and a conclusory step 140c of connecting a bag valve respirator to the tubing. Further, the activity identification engine 106 may analyze the image data 104b in view of subsequent activity (e.g., successful respirations, removal of the tubing, etc.) to determine completion and/or failure of the medical procedure 140.

In some implementations, through use of the context information, a different subset of the procedure models 108 may be applied than were used in the situation described in relation to FIG. 1B. For example, a portion of the procedure models 108 may be context-enhanced models that, through relying on context information to key in on body areas, types of medical procedures, and/or a reduced set of steps to identify, require less processing capacity and/or memory space to perform recognition analysis. The context-enhanced models, for example, may provide the benefit of being executable in real-time or near real-time on a variety of computing devices (e.g., off-the-shelf tablet and/or laptop computers, etc.) rather than requiring an edge server or computing farm for intensive processing.

In some embodiments, the procedure identifier(s) 110, rather than and/or in addition to being provided to the ePCR coding conversion engine 112 of FIG. 1B, are provided to a billing category conversion engine 134 for translating the procedure identifier(s) 110 into one or more invoice codes 136 for invoicing purposes. The billing category conversion engine 134, for example, may correlate the procedure identifiers 110 to invoice codes 136 using a procedure mappings to invoice codes data set 138 (e.g., mapping table, etc.). The billing categories, in some examples, may include basic life support (BLS), advanced life support (ALS), ALS level 1 (ALS1), and/or ALS level 2 (ALS2). In illustration, the ALS1 billing category may include the following medical procedures: 3-Lead EKG, 12-Lead EKG, advanced life support (ALS) assessment, cardiac monitor, cardioversion, central intravenous (IV), cricothyrotomy, defibrillation, external pacing, urinary catheter (e.g., an indwelling catheter such as a Foley® catheter or an intermittent catheter), internal pacing, Intraosseous (IO) access, nasotracheal intubation, needle thoracotomy, needle thoracostomy, nasogastric (NG) tube, peripheral IV, orotracheal, orogastric (OG) Tube, or SIO. In comparison, the ALS2 billing category may include the following medical procedures: manual defibrillation/cardioversion, Defibrillation, Cardioversion, Endotracheal intubation, combitube, nasotracheal intubation, orotracheal, central venous line, central IV, cardiac pacing, external pacing, internal pacing, chest decompression, chest tube, surgical airway, needle cricothyrotomy, needle thoracotomy, orotracheal, surgical cricothyroidotomy, Intraosseous line, or IO access. Where this is overlap between procedures (e.g., procedures that appear as both ALS1 and ALS2), the level determination may depend on the person performing the procedure, the degree to which the caregiver may adjust parameters for the procedure, the particular equipment used for the procedure, the type of injury or illness being treated, the medical necessity, teleguidance or other supervision, etc.

The billing categories may depend in part on a skill level and/or certification level of the caregiver performing the medical procedure. Further, the billing categories may depend in part on a type of equipment used for performing the medical procedure.

In some implementations, the invoice code(s) 136 determined by the billing category conversion engine 134 are stored as invoice data 145. The invoice data 145, for example, may be shared with a medical billing system, as discussed in relation to FIG. 9, below.

FIG. 1D is an operational flow diagram of an example process 150 for automatically identifying an emergency medical procedure 170 and assessing quality of procedure performance using image data analysis. Aspects of the example process 150 may be used in combination with elements of the process 100 of FIG. 1B and/or the process 130 of FIG. 1C. However, unlike the process 100 of FIG. 1B and the process 130 of FIG. 1C, evaluating performance of medical procedures may be conducted days, weeks, or even months after the medical procedure 170 was performed, assuming image data 104c was archived for the purpose. For example, performance assessments may be conducted periodically to rate or rank caregivers and/or to designate a level of familiarity of each caregiver with certain medical procedures. Conversely, the process 150 may be performed in real-time or near real-time, in some embodiments, to identify whether a medical procedure was successfully performed (e.g., for ePCR logging purposes and/or invoicing purposes).

In some implementations, the process 170 begins with a precursor step identification engine 152 analyzing the image data 104c to identify activity that is the precursor to performing one or more medical procedures. The precursor step identification engine 152, for example, may analyze one or more incoming streams of image data for evidence of a precursor step being performed. Image data, for example, may be temporarily buffered during analysis and, if no precursor step is discovered, discarded (e.g., overwritten in a circular buffer, etc.). In this manner, the precursor step identification engine 152 acts as an initial screening operation for determining whether a medical procedure is about to be performed. In analyzing the image data 104c, in some embodiments, the precursor step identification engine 152 analyzes lower quality image data, such as compressed image data or wireframe image data, for example to increase processing speed and/or decrease resource use (e.g., memory, battery, and/or processor cycles required). The image data supplied to the precursor step identification engine 152, in some embodiments, includes image data from a portion of the image capture devices 102. For example, the precursor step identification engine 152 may focus on object movements as captured in wireframe data supplied by the LiDAR sensor(s) 102c. The precursor step identification engine 152, in some embodiments, causes additional image capture devices, such as the device camera(s) 102a and/or the wearable camera(s) 102b, to actively collect and provide image data responsive to identifying a precursor step.

The precursor step identification engine 152, in some implementations, analyzes the image data 104c in view of a subset of procedure models 108a (e.g., procedure models designed to identify common precursor actions). The precursor activities, in some examples, can include unwrapping a medical equipment item, attaching a therapy device to the patient, and/or preparing a section of the patient's body (e.g., cleaning skin, positioning head angle, etc.). The procedure models 108a, for example, may be trained to identify a first step of one or more activity workflows corresponding to one or more medical procedures. Each activity workflow, in turn, may be represented by one or more additional procedure models 108b. Each activity workflow, for example, encompasses a series of steps executed in performing a medical procedure.

In some implementations, upon identifying a precursor step of one or more medical procedure activity workflows, the precursor step identification engine 152 provides an activity identifier 154 to a procedure monitoring engine 156 to analyze the image data 104c in view of the activity identifier 154. Using the activity identifier 154, for example, the procedure monitoring engine 156 may select one or more procedure models of the collection of procedure models 108b corresponding to the one or more medical procedures having the precursor step identified by the activity identifier 154. As described in relation to FIG. 1B, the selected models may further correspond to a particular type of image data. Further, as described in relation to FIG. 1C, the selected models may correspond to availability or unavailability of context data.

Using the one or more procedure models, in some implementations, the procedure monitoring engine 156 identifies a series of steps of the medical procedure 170. As illustrated, the medical procedure is a procedure for supporting the ventilations of a patient. The steps of the medical procedure 170 include assembling the oxygen supply 170a, attaching an oxygen regulator to an oxygen tank and adjusting the flow rate 170b, insertion of an oropharyngeal airway (OPA) 170c, and positioning the mask over the mouth and nose of the patient. The procedure monitoring engine 156 may be configured to recognize a beginning of each step and calculate a length of time to accomplish each step and/or the medical procedure 170 as a whole, from accessing the medical equipment items (e.g., bag valve mask, OPA, oxygen tank, etc.), to positioning the medical equipment items, to ensuring successful respirations. In other examples, the procedure for supporting the ventilations of a patient could include connection of the oxygen supply to a nasal cannula or non-rebreather mask instead of the bag valve mask. Additionally, as discussed above, the procedure for supporting the ventilations of a patient may include selection of a properly sized OPA or selection and use of a nasopharyngeal airway (NPA) instead of an OPA. The procedure monitoring engine 156 may be configured to recognize an endpoint of one or more steps, such as removal of a hand of the caregiver from the OPA being indicative of completion of placement. Further, the procedure monitoring engine 156 may recognize and log repetitions of steps (e.g., accidentally dropping the OPA, accessing a new OPA, etc.). Repetition could be indicative of failure of performance on a first attempt.

Further, as discussed in relation to the context data 132 of FIG. 1C, in some implementations, the procedure monitoring engine 156 identifies a caregiver performing the medical procedure. In the circumstance of performing staff evaluations, for example for gauging training opportunities and/or for reassessing proficiency in performing the medical procedures, the process 150 may include associating the performance evaluation with a particular caregiver (or caregivers if multiple caregivers are involved). In the circumstance of multiple caregivers, each step identified may be associated with a particular caregiver to the extent possible considering image data quality and/or scene obstruction.

After final positioning at step 170c, the procedure monitoring engine 156, in some embodiments, monitors further image data and/or context data to confirm success of the operation. Confirming success, in the example of respiration assistance using a bag valve mask, may include monitoring sensor data from the bag valve mask, evaluating physiological data provided by one or more medical devices and/or identified through image analysis, and/or analyzing image data to confirm maintenance of the bag valve mask positioning for at least a threshold period of time (e.g., thirty seconds, one minute, etc.). Registering failure, conversely, may include monitoring for removal of the bag valve mask within a threshold period of time.

In some implementations, the procedure monitoring engine 156 provides step identifiers and timings 158 (and, in some circumstances, context data such as identified caregiver(s)) for the medical procedure 170 to a procedure quality analysis engine 160 for calculating performance metrics associated with performance of the medical procedure. The procedure quality analysis engine 160, for example, may calculate performance metrics, such as timing metrics, waste metrics, and/or success metrics in comparison to a benchmark and/or target performance level. Further, the procedure quality analysis engine 160 may compare the series of step identifiers 158 to a procedure workflow and/or a procedure protocol to ensure adherence to best practices.

In some implementations, one or more of the activity identification engine 154, the procedure monitoring engine 156, and the precursor step identification engine 152 may include or be coupled to a procedure guidance engine 180. The procedure guidance engine 180 may include or be coupled to a guidance library 185. During patient care, when one or more of the activity identification engine 106, the procedure monitoring engine 156, and the precursor step identification engine 152 predict, identify, or monitor an activity, step, or procedure, these engines may invoke the procedure guidance engine 180. The procedure guidance engine 180 may generate caregiver guidance 190 based on a guidance library 185 where the caregiver guidance 190 corresponds to the activity, step, or procedure. In the example shown in FIG. 1D, the caregiver guidance 190 is guidance for a selection of a properly sized oropharyngeal airway (OPA), for example the OPA shown at 170c. The procedure guidance engine 180 may provide the caregiver guidance 190 automatically or may provide a prompt for a caregiver to request the guidance. The procedure guidance engine 156 may provide the guidance and/or the prompt visually and/or audibly for example at the wearable camera device 102b, at a device providing the patient charting application 116, or another caregiver interface device 306. The OPA sizing shown for example in FIG. 1D is also another example of a precursor step that may be identified by the precursor step identification engine 152. Preparation steps may also enable the engines 106, 152, and/or 156 to distinguish between similar procedures. For example, an OPA insertion is similar in many regards to a nasopharyngeal airway (NPA) insertion in the sense that both are a respiratory intervention, both require a measurement, both involve introduction of a device at the face of a patient, etc. However, the proper identification of these procedures may be critical for recognizing the state of the patient and the level of care provided. For example, in general an NPA is used in a conscious patient and an OPA is reserved for an unconscious patient to avoid the gag reflex. The difference between an unconscious and conscious patient may be important for activity recognition and recordation as well as for contextual information for other care activities.

In some implementations, the activity identification engine 106, the procedure monitoring engine 156, and/or the precursor step identification engine 152 include caregiver guidance materials as contextual information used to determine a confidence in predictive or identification output. The caregiver guidance materials may be those selected by the guidance engine 180 and/or guidance materials selected and reviewed independently from the guidance engine 180 or in the absence of such an engine. For example, the caregiver may select materials without assistance from a guidance engine 180. In addition to reference materials, such contextual information may further include alerts or decision support protocols provided at a caregiver interface device 306 and/or a medical device (e.g., the medical devices 932).

In some implementations, the procedure quality analysis engine 160 generates one or more outcomes and/or ratings 162. The outcomes, in some examples, may include success/failure and/or patient health status (e.g., improved/declined/deceased). The ratings, in some examples, may include adherence to protocol (e.g., correct/incorrect, percentage adherence, level of adherence such as unsatisfactory/satisfactory/exemplary, etc.), level of waste (e.g., excess medical equipment items used), and/or at least one length of time rating regarding certain steps and/or the procedure as a whole (e.g., faster than benchmark, close to benchmark, slower than benchmark, etc.).

Although not illustrated, in other implementations, the procedure quality analysis engine 160 may obtain the image data 104c for quality analysis. For example, the procedure quality analysis engine 160 may be configured to assess caregiver management of the medical procedure such as, in some examples, caregiver demeanor (e.g., tone and/or communication derived from corresponding audio portion of the data, demeanor derived from body movements/facial expressions, etc.), caregiver handling of patient (e.g., rough, careful, etc.), and/or caregiver handling of medical equipment (e.g., attention to avoiding contamination, attention to avoiding injury to self and/or patient, etc.). The procedure quality analysis engine 160 may generate one or more management ratings and/or metrics related to the image analysis performed.

In some implementations, the outcomes and/or ratings 162 are stored as procedure review data 164. If the procedure quality analysis engine 160 is provided with context information, the procedure quality analysis engine 160 may correlate the procedure review data 164 with a portion of the context such as, in some examples, a caregiver, a caregiver team, a date and/or time, identification of an emergency medical vehicle, and/or patient medical information.

Although not illustrated, in further implementations, the procedure review data 164, the step identifiers and timings 158, the image data 104c, and/or certain context data may be archived for procedure model training purposes. For example, the training of one or more procedure models 108 corresponding to the medical procedure 170 may be refined using the data. Further, one or more context-enhanced procedure models 108 may be created using context data collected during the medical emergency event.

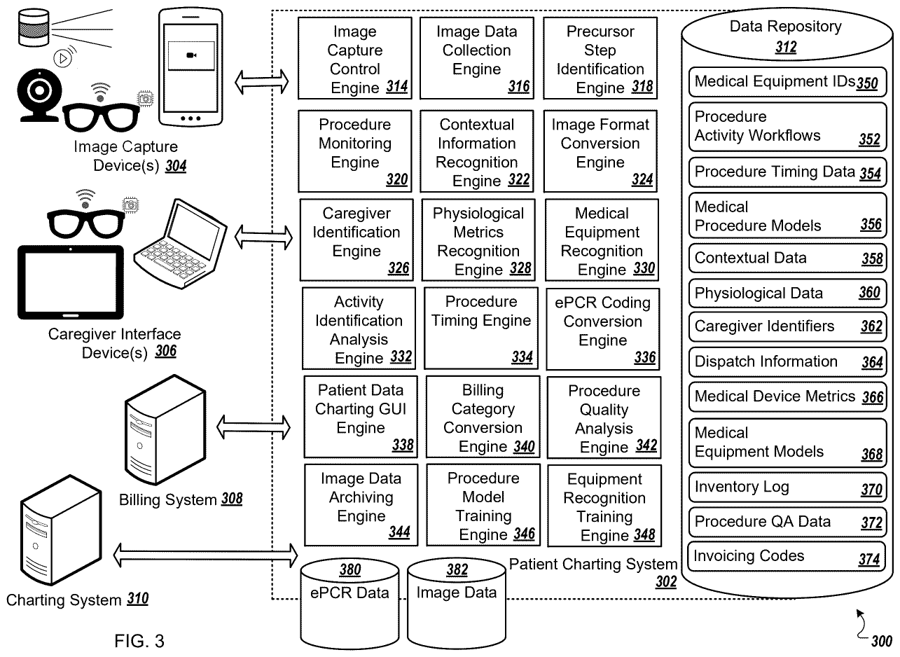

Turning to FIG. 3, a block diagram illustrates an example patient charting system 302 and environment 300. The patient charting system 302, for example, may be used to perform the example processes of FIG. 1B through FIG. 1D. As illustrated, the environment includes image capture devices 304 (e.g., still cameras, video cameras, three-dimensional video cameras, LiDAR sensors, three-dimensional LiDAR sensors, etc.), such as the devices 102 of FIG. 1B through FIG. 1D and/or the devices 202 of FIG. 2, in communication with the patient charting system 302 to supply image data 382. Further, the environment 300 includes a set of caregiver interface device(s) 306, such as the tablet device 202a of FIG. 2 or the wearable device 102b of FIG. 1A, or another caregiver interface device 306. The caregiver interface device(s) 306, for example, may be used to enter ePCR data 380 that is not automatically captured through image analysis. As illustrated in FIG. 2, one or more of the caregiver interface device(s) 306 may also function as one of the image capture device(s) 304. Additionally, the patient charting system 302, as illustrated, is in communication with an external billing system 308 and an external charting system 310. The patient charting system 302 is configured to access information from a data repository 312.

The image capture device(s) 304 and/or the caregiver interface device(s) 306 may be disposed throughout an emergency medical scene. The image capture device(s) 304 (e.g., cameras and/or LiDAR sensors), in some examples, may include one or more handheld devices and/or wearable devices, as illustrated, for example, in FIG. 2. The wearable devices may include smart glasses devices, smart watch devices, and/or body cameras (e.g., body camera 202c of FIG. 2). As illustrated by the defibrillator 202b of FIG. 2, one or more image capture devices 304 may be integrated into an EMS equipment item, such as a medical device, patient monitoring device, computing device (e.g., such as the tablet computer 202a of FIG. 2), and/or carrying case. In another example, one or more image capture devices 304 may be configured to mount to an EMS equipment item, such as a gurney, patient monitoring device, or carrying case. In some embodiments, one or more image capture devices 304 may be integrated into or mounted to an interior of an emergency transport vehicle.

To enable capture of medical procedures at the emergency medical scene no matter how the caregivers are positioned and/or no matter what area of the patient's body is being worked on, in some implementations, one or more of the image capture devices 304 may be mounted to or integrated into a remotely controllable movable mount. The remotely controllable movable mount, for example, may follow movement of objects, such as medical equipment items and/or caregivers to focus on a region where the medical procedure is being performed. In another example, the remotely controllable movable mount may track a position of the patient and/or at least one caregiver. Different remotely controllable movable mounts may be programmed to focus on different targets (e.g., one patient-focused mount, one caregiver-focused mount, etc.). The remotely controllable movable mount, in some examples, may include a gimbal, a remotely controllable drone, and/or a mount configured to allow limited travel in two-dimensional or three-dimensional space (e.g., a rail system on a roof of the medical transport vehicle, a wheeled mount configured to travel along a surface, etc.).

In some implementations, an image capture control engine 314 controls capture of the image data 382 from the image capture device(s) 304. The image capture control engine 314, for example, may activate one or more image capture device(s) 304 responsive to an audible command provided by a caregiver and/or another audible cue such as, in some examples, the sound of a piece of medical equipment powering up (e.g., a recognizable chirp), a sound of return of caregivers to the emergency medical transport after reaching the scene of the medical emergency, and/or the sound of the emergency transport vehicle doors opening (e.g., indicative of arrival at scene). In another example, the image capture control engine 314 may activate one or more image capture device(s) 304 responsive to detecting presence of a patient. In some examples, the patient may be detected by one or more sensors (e.g., weight sensors, motion sensors, etc.) of a gurney or other transportation surface or treatment surface on which a patient would be positioned. In a further example, the image capture control engine 314 may activate one or more image capture device(s) 304 responsive to detecting arrival at the emergency medical scene. For example, a mapping system and/or GPS system of the emergency transport vehicle may be used to indicate arrival, or a caregiver may log arrival in the patient charting system 302 or other caregiver data collection system. In an additional example, the image capture control engine 314 may activate one or more image capture device(s) 304 responsive to detecting motion, such as a motion detector positioned at a rear of the emergency medical vehicle and/or a motion detector positioned proximate to a patient positioning surface (e.g., gurney, treatment surface, etc.).

The image capture control engine 314, in some implementations, activates one or more image capture device(s) 304 based on inputs from another of the image capture device(s) 304. A first image capture device 304 may be capturing image data to detect activity at the emergency scene, such as motion of medical equipment and/or caregivers. The first image capture device 304, for example, may be a LiDAR sensor configured to capture wireframe data and monitor the wireframe data for movement at the scene. Responsive to the first image capture device 304 detecting activity, the image capture control engine 314 may be alerted, thereby activating further image capture device(s) 304 and beginning to collect the image data 382 for analysis. For example, the image capture control engine 314, upon recognizing activity at the emergency medical scene, may activate an image data collection engine 316.

In some embodiments, the image data collection engine 316 collects data from each active image capture device 304. The image data collection engine 316, for example, may collect the image data 382 in a temporary cache area for initial analysis by one or more of a precursor step identification engine 318, a procedure monitoring engine 320, a contextual information recognition engine 322, and/or an image format conversion engine 324.

In some implementations, the precursor step identification engine 318 analyzes the image data 382 to identify a precursor to performing one or more medical procedures (e.g., in accordance with one of a set of procedure activity workflows 352, as discussed in relation to FIG. 1D). The precursor step identification engine 318, for example, may perform operations as described in relation to the precursor step identification engine 152 of FIG. 1D. Upon identifying the precursor step, for example, the procedure monitoring engine 320 may be activated to monitor for performance of the medical procedure.

In some implementations, the procedure monitoring engine 320 monitors a series of steps performed by one or more caregivers to identify correspondence to one of the set of procedure activity workflows 352 and/or a medical procedure protocol. The procedure monitoring engine 320, for example, may be configured to perform operations as described in relation to the activity identification engine 106 of FIG. 1B and FIG. 1C and/or the procedure monitoring engine 156 of FIG. 1D. In some implementations, the procedure monitoring engine uses an activity identification engine 332 (e.g., such as the activity identification engine 106 of FIG. 1B and FIG. 1C) to identify the series of steps of the medical procedure as well as the contextual information recognition engine 322 to identify context associated with the medical procedure (e.g., such as the context data 132 described in relation to FIG. 1C) and/or a procedure timing engine 334 to collect timing information regarding a length of time of steps of the medical procedure and/or the procedure itself (e.g., as described in relation to the procedure monitoring engine 156 of FIG. 1D). The procedure timing engine 334 may save timing information as procedure timing data 354.

In some implementations, the procedure monitoring engine 320 and/or the activity identification engine 332 apply a collection of medical procedure models 356 to analyzing the image data 382 to recognize the series of steps of one of the procedure activity workflows 352. For example, the medical procedure models may be applied as described in relation to the procedure models 108 of FIG. 1B through FIG. 1D.

In some implementations, the procedure monitoring engine obtains contextual data 358 from a contextual information recognition engine 322. The contextual information recognition engine 322, for example, may analyze image data captured prior to performance of the medical procedure and/or during performance of the medical procedure to identify various contextual elements in the image data 382 such as, in some examples, patient physiological data 360, caregiver identifiers 362, and/or medical equipment identifiers 350. The contextual information recognition engine 322, for example, may interoperate with a caregiver identification engine 326, a physiological metrics recognition engine 328, and/or a medical equipment recognition engine 330 to collect the contextual data 358.

In some implementations, the caregiver identification engine 326 is configured to recognize particular caregivers based on contextual information in the image data 382. The contextual information, in some examples, can include facial recognition, badge recognition (e.g., NLP of text, scanning of a machine-readable code, etc.), and/or identification of caregiver identifiers in dispatch information 364 supplied to the emergency medical vehicle, as described in relation to the context data 132 of FIG. 1C.

In some implementations, the physiological metrics recognition engine 328 recognizes physiological metrics (e.g., from medical device displays, etc. captured in the image data 382) and/or derives physiological metrics from analyzing images taken of the patient, as described in relation to the context data 132 of FIG. 1C. Further, the physiological metrics recognition engine 328 may obtain a portion of the physiological metrics from stored medical device metrics 366 captured by one or more medical devices.

In some implementations, the medical equipment recognition engine 330 analyzes at least a portion of the image data 382 to recognize medical equipment (e.g., by medical equipment identifiers 350) used for medical procedures. The medical equipment recognition engine 330, for example, may analyze markings on medical equipment items as described in relation to the context data 132 of FIG. 1C. The medical equipment recognition engine 330, further, may apply one or more trained medical equipment models 368 to recognizing images of commonly used medical equipment items.

In some implementations, the medical equipment recognition engine 330 performs inventory analysis on medical equipment items visible to the image capture device(s) 304. For example, the medical equipment recognition engine 330 may generate an object map of various medical equipment items within an emergency medical scene, such as a region of a medical transport vehicle. A LiDAR sensor image capture device 304, for example, may be used to create a wireframe map of the medical equipment items, tagging each item by medical equipment identifier 350 and recognizing movement of any of the medical equipment items (e.g., due to being used by caregivers to perform medical procedures). The recognized movements, for example, may be used by the precursor step identification engine 318 to recognize a precursor to a medical procedure (e.g., accesses one or more particular medical equipment items). The inventory may be captured in an inventory log 370.

In some implementations, prior to the image data 382 being analyzed, the image format conversion engine 324 converts at least a portion of the image data 382 into an appropriate format for analysis. The image format conversion engine 324, for example, may align LiDAR sensor data with camera data for use by the medical equipment recognition engine in generating the object map of the medical equipment items. The image format conversion engine 324, in another example, may co-register captured angles of the image data 382 to generate three-dimensional camera/video data and/or three-dimensional LiDAR data (e.g., 3D point cloud data). In a further example, the image format conversion engine 324 may compress a portion of the image data 382 to reduce size and/or complexity of the data analyzed. In illustration, the image format conversion engine 324 may reduce a number of colors in a portion of the image data 382.

In some implementations, an output of the procedure monitoring engine 320 or activity identification engine 332 identifying the medical procedure (e.g., by a medical procedure identifier) is provided to an ePCR coding conversion engine 336 to convert the information regarding the identified medical procedure to ePCR field data for storing as ePCR data 380. The ePCR coding conversion engine 336, for example, may perform operations described in relation to the ePCR coding conversion engine 112 of FIG. 1B.

In some implementations, prior to logging the ePCR data (e.g., ePCR field data 114 of FIG. 1B) to the ePCR data 380 and/or prior to uploading the ePCR data to the charting system 310, the caregiver(s) at the scene may be prompted, via a patient data charting graphical user interface (GUI) engine 338, for confirmation regarding the identified medical procedure information. The patient data charting GUI engine 338, for example, may present the information prepared for logging as ePCR data to one of the caregiver interface device(s) 306 for confirmation.

In some implementations, an output of the procedure monitoring engine 320 or activity identification engine 332 identifying the medical procedure (e.g., by a medical procedure identifier) is provided to a billing category conversion engine 340 for generating invoicing codes 374 corresponding to the medical procedure performed. For example, the billing category conversion engine 340 may perform operations described in relation to the billing category conversion engine 134 of FIG. 1C. The billing category conversion engine 340, for example, may provide the invoicing codes 374 to the billing system 308.

In some implementations, an output of the procedure monitoring engine 320 identifying steps and timings related to performance of the medical procedure is provided to a procedure quality analysis engine 342 for generating procedure quality assurance (QA) data 372 related to performance of the medical procedure. The procedure quality analysis engine 342, for example, may perform operations described in relation to the procedure quality analysis engine 160 of FIG. 1D.

In some implementations, as described in relation to FIG. 1D, the procedure quality analysis engine 342 analyzes archived image data 382. An image data archiving engine 344, for example, may collect image data to a long-term data storage for later use. The image data archiving engine 344, in some implementations, archives portions of the image data 382 identified as capturing performance of a medical procedure (e.g., by the precursor step identification engine 318, procedure monitoring engine 320, and or the activity identification engine 332). In some embodiments, the image data archiving engine 344 archives context data 358, medical device metrics 366, physiological data 360, procedure timing data 354, invoicing codes 374, medical equipment identifiers 350, dispatch information 364, and/or ePCR data 380 corresponding to the image data 382 along with the image data 382. The archived data, for example, may be used for refining training of the medical procedure models 356 and/or for producing new medical procedure models.

In some embodiments, the image data archiving engine 344 transforms the image data prior to archiving. For example, the image data archiving engine 344 may convert the image data to a form that obscures identifying features, such as, in some examples, facial features, tattoos, medical bracelet information, and/or medical chart information visible in the image data, that could otherwise be used to uniquely identify the patient. For example, the image data archiving engine 344 may apply blurring or an overlay to portions of the image data. In another example, the image data may be converted to a compressed or modified format that would naturally obscure identifying features, such as a wireframe data format.

The image data archiving engine 344, in some embodiments, archives the image data to an external archival region. The external archival region, for example, may be located in the cloud, on an edge server, and/or in a networked storage region connected to a remotely located server. Prior to transferring the image data, the image data archiving engine 344 may compress the image data to reduce time of transit.

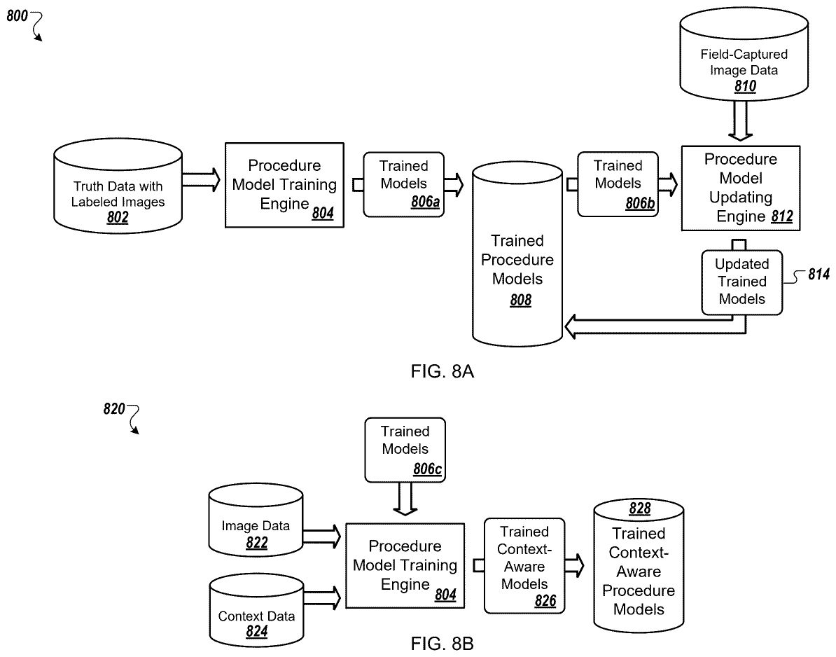

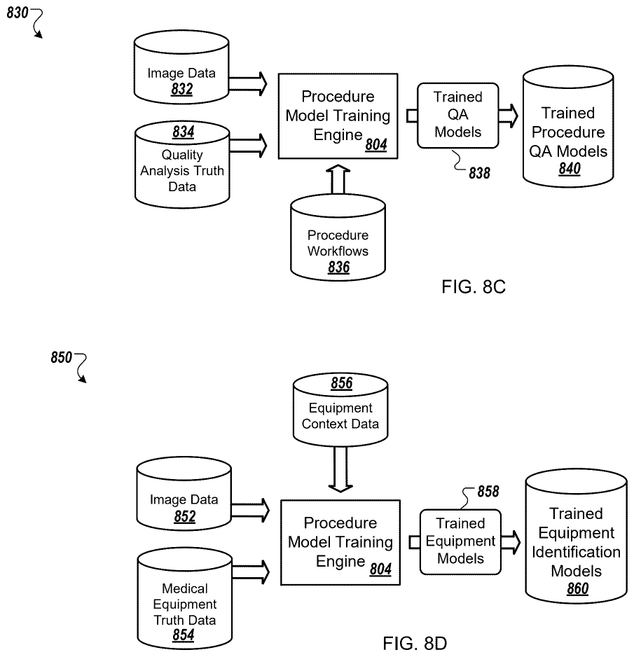

In some implementations, a procedure model training engine 346 is configured to access archived data and to update the training of the medical procedure models 356. In some embodiments, the procedure model training engine 346 creates one or more new medical procedure models using the archived data. The new procedure models, for example, may include context-aware procedure models that apply context data (e.g., the contextual data 358, physiological data 360, dispatch information 364, medical equipment identifiers 350, invoicing codes 374, and/or ePCR data 380) to simplify the processing intensiveness and/or memory requirements of a corresponding medical procedure model 356 trained with no context data or fewer/different types of context data. Training is described in greater detail below in relation to FIG. 8A through FIG. 8D.

In some implementations, an equipment recognition training engine 348 is configured to train one or more medical equipment models 368 to recognize medical equipment used to perform the collection of medical procedures. The equipment recognition training engine 348 may be supplied initial truth data, in one example, using image data capturing visually-tagged medical equipment items. The visual tags, in some examples, can include labels (e.g., stickers) having differing colors and/or shapes, bar codes, QR codes, and/or initial placements/holders being affixed with the visual tags (e.g., a bag of gauze, a box of disposable gloves, etc.). Using the truth data, the equipment recognition training engine 348 may be trained to recognize various sizes, models, manufacturers, and/or versions of medical equipment items used in the medical procedures.

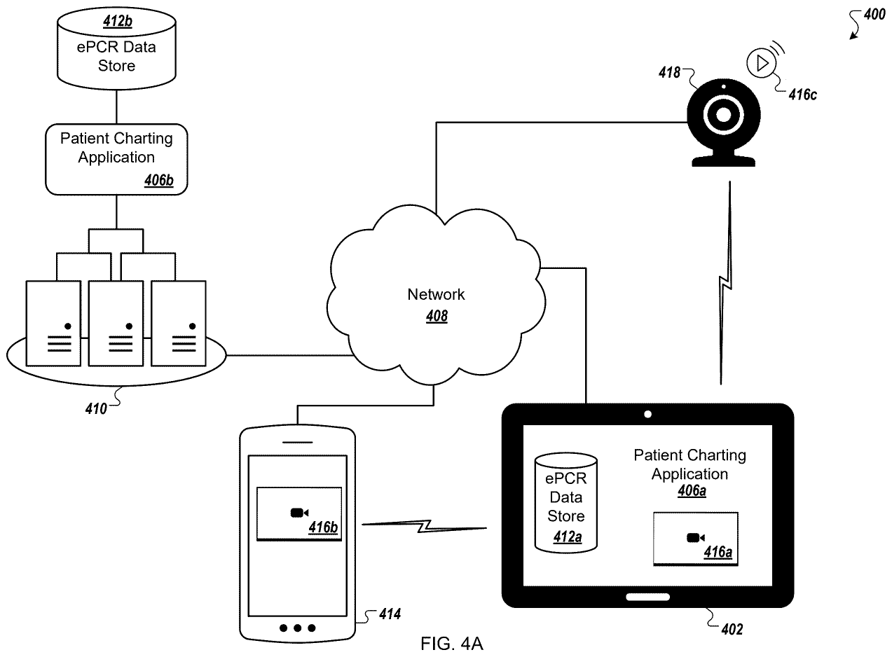

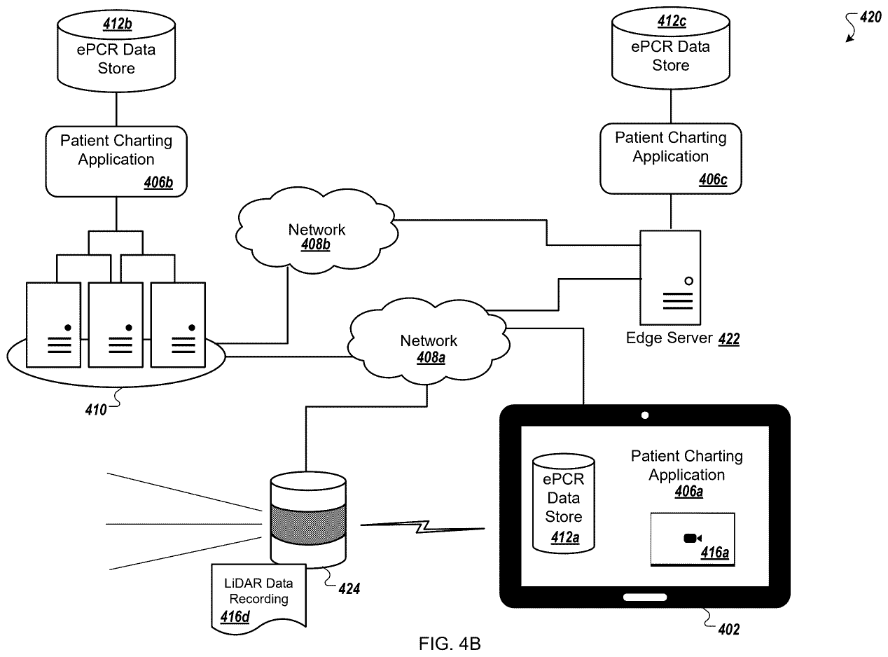

FIG. 4A illustrates an example system 400 for automatically populating ePCR data based on analysis of medical procedures captured in image data. As shown in FIG. 4A, the system 400 includes a smartphone 414, a tablet computing device 402, a network 408, and a server environment 410. The tablet 402 hosts a patient charting application 406a and an ePCR data store 412a. The server environment 410 hosts a patient charting application 406b and an ePCR data store 412b. The smartphone 414 may be configured to connect to the tablet 402 via a short-range wireless connection (e.g., a personal area network (PAN) connection, such as a Bluetooth connection, or a local area network (LAN) connection, such as a Wi-Fi connection) and to the network 408 via a long-range wireless connection (e.g., a wide area network (WAN) connection, such as a Code-Division Multiple Access (CDMA) connection or Global System for Mobile Communication (GMS) connection). Similarly, the tablet 402 may be configured to connect to the smartphone 414 via a short-range wireless connection, such as a PAN connection or LAN connection, and to the network 408 via a long-range wireless connection, such as a WAN connection.

In some implementations, the tablet 402 is pre-configured to be associated with a medical treatment diagnostic device and/or edge server so as to streamline wireless communication pairing without having to undergo a time-consuming inquiry and response negotiation for a secure connection to be established. The tablet 402 may be a companion device of a medical treatment and/or diagnostic device. In some embodiments, the companion device is dedicated to communicating only with its corresponding medical and/or diagnostic device. In some embodiments, the companion device can display sensor data in real-time from one or more physiological sensors connected to the medical treatment device. The companion device, for example, may be configured to display a visual reproduction of the information displayed at the medical treatment device in a first display. The visual reproduction, for example, may encompass an exact replication of the data displayed at the medical treatment device. In another example, the visual reproduction may include data and formatting variations that can enhance viewing and comprehension of the case information by the companion device user. The display layout, magnification of each data section, physiologic waveform selection, physiologic numeric readout selection, resolution, waveform duration, waveform size, text size, font, and/or display colors, in some examples, may vary from what is displayed at the medical treatment device(s).

The server environment 410, which includes one or more physical and/or virtual servers, in some implementations, is configured to connect to the network 408 via a robust network connection, such as a dedicated and redundant service provider connection. The network 408, for example, may be a high-availability public or private network, such as the Internet, through which computing devices exchange (transmit and/or receive) communications. In certain embodiments, computer-implemented processes described herein interoperate over the connections described above via one or more application programming interfaces (APIs) implemented by the processes.

The charting applications 406a and 406b and the data stores 412a and 412b may be configured to operate collaboratively or independently, depending on the design goals of a particular installation. For example, in some implementations, the charting application 406b serves the charting application 406a as a browser-based user interface to the tablet 402. In such implementations, the charting application 406a may be a thin client relying on periodic communications with the charting application 406b to operate properly. Moreover, in such embodiments, the data store 412a may be maintained in browser session storage and, thus, may contain a limited amount of data that is updated periodically with data from the data store 412b. Alternatively or additionally, in some embodiments, the charting application 406a is an independent application configured to execute natively under an operating system of the tablet 402. In such embodiments, the data store 412a may contain all of the data needed for the charting application 406a to operate properly. In either case, it should be noted that the data stores 412a and 412b may exchange information periodically or in real-time to maintain data currency.

In some implementations, the charting application 406a obtains image recordings 416b from the smartphone 414 and/or image recordings 416c from an image capture device 418. The tablet device 402 on which the patient charting application is executing, further, may obtain image recordings 416a. The patient charting application 406a may be configured to analyze the image recordings 416 as described in relation to the procedure monitoring engine 320 and/or the activity identification engine 332 of FIG. 3 to augment ePCR data of the data store 412a.

In some implementations, for example where the processing requirements are too intensive for the tablet device 402 and/or when a reliable connection is available to the server environment 410 via the network 408, the tablet device 402, the smartphone 414, and/or the image capture device 418 may provide the image recordings 416 to the server environment 410 via the network 408 for use by the patient charting application 406b.

Turning to FIG. 4B, in some implementations, a system 420, in addition to many of the same elements as the system 400 of FIG. 4A, includes an edge server 422. The edge server 422, for example, may be a computing device configured to execute processor intensive operations that are sometimes involved when executing machine learning processes, such as artificial intelligence computer vision operations. The edge server 422 may include, for example, one or more graphic processing units (GPUS) that are capable of efficiently executing matrix operations as well as substantial cache and/or other high-speed memory to service the GPUs. In some embodiments, the edge server 422 is a separate, ruggedized physical device that travels with EMS personnel in the field. In some embodiments, the edge server 422 is incorporated into other EMS field equipment such as a medical device and/or may be located in or integrated into the EMS vehicle. Alternatively or additionally, the edge server 422 may be located within a carrying case for a medical device. In some embodiments, a portable computing device such as a smart phone or tablet may operate as the edge server 422, for example if the processing capability of these devices is sufficient to provide computing services associated with the edge server 422. The edge server 422 may be a local device to the tablet 402 (e.g., located in proximity to one another and to the EMS personnel and/or the emergency victim). The server environment 410, conversely, may be hosted in a cloud service including one or more cloud servers located remote from the edge server 422 and other devices (e.g., tablet 402). The edge server 422, for example, may be configured to move more computing capability into the local environment so that the computation intensive image models can run accurately and efficiently to support the patient charting application 406 even in the absence of connection with the remote cloud server 410. In some embodiments, the tablet 402 lacks the processing capability necessary to support at least a portion of the models.

Regardless of physical form, the edge server 422, in some implementations, is configured to interoperate with other devices of the system 420 directly or via the network 408. For instance, the edge server 422 may include a wireless network interface (e.g., a PAN interface, LAN interface, WAN interface, or the like) through which the edge server 422 can communicate with the tablet 402, an image capture device (e.g., a LiDAR sensor 424, the image capture device 418 of FIG. 4A, and/or the smartphone 414 of FIG. 4A), and/or the server environment 410. In a reciprocal manner, the image capture device(s) and/or the tablet 402 may be configured to connect directly or indirectly to, and interoperate with, the edge server 422, via a short-range wireless connection, such as a PAN connection or a LAN connection. In some embodiments, the image capture device(s) and/or the tablet 402 may communicate via a short-range wireless network connection (e.g., network 408a) to the edge server 422 and, in turn, the edge server 422 may communicate via a long range wireless connection (e.g., network 408b) to the server environment 410. The computer-implemented processes described herein, in some embodiments, are configured to interoperate with one another over the connections described above, via one or more APIs implemented by the processes.

The additional computing resources provided by the edge server 422, in some implementations, add several capabilities to the system 420. For example, the edge server 422 may enable the tablet 402 to tolerate faults and operate robustly in the face of an inoperable WAN connection to the server environment 410. For instance, the patient charting application 406a may be configured to interoperate with the patient charting application 406c by default, and the patient charting application 406c or the data store 412c may be configured to replicate data from the data store 412c to the data store 412b when an operable WAN connection to the server environment 410 is available. This implementation tolerates WAN connection faults well because the patient charting application 406c can store ePCR data in the ePCR data store 412c for extended WAN connection outages periods and relay the stored ePCR data to the ePCR data store 412b when the WAN connection becomes available. Other approaches to establishing a high-availability and fault-tolerant system that the edge server 422 enables will be apparent in view of this disclosure. In illustration, the edge server 422 may operate as a proxy server designed to failover from the patient charting application 406b to the patient charting application 406c upon detection of a WAN connection fault.

Other advantages realized via the edge server 422 include faster and more accurate execution of machine learning processes and lower latency in data availability between instances of the patient charting application 406. These benefits are realized by virtue of the edge server's powerful hardware and central storage and synchronization of ePCR data.

It should be noted that some implementations of the system 400 can be configured to convert to the system 420 upon introduction and detection of the edge server 422 by any of the processes/devices of the system 400.

In some implementations, a LiDAR sensor device 424 collects LiDAR data recordings 416d and transfers the LiDAR data recordings 416d to the patient charting application 406a, the patient charting application 406b, and/or the patient charting application 406c.

In some implementations, the charting application 406a obtains the LiDAR data recordings 416d from the LiDAR sensor 424 for local processing, for example via a local wireless network. The patient charting application 406a may be configured to analyze the image recordings 416 as described in relation to the procedure monitoring engine 320 and/or the activity identification engine 332 of FIG. 3 to augment ePCR data of the data store 412a.

In some implementations, for example where the processing requirements are too intensive for the tablet device 402 and/or when a reliable connection to the server environment 410 is unavailable via the network 408a, the tablet device 402 and/or LiDAR sensor 424 may provide the image recordings 416a, 416d to the edge server 422 via the network 408a for use by the patient charting application 406c.

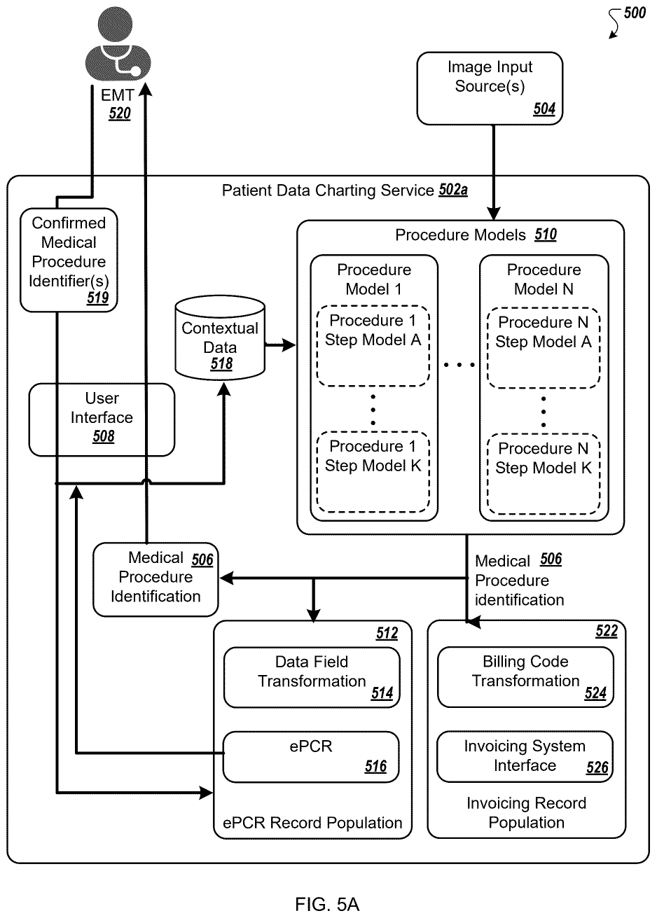

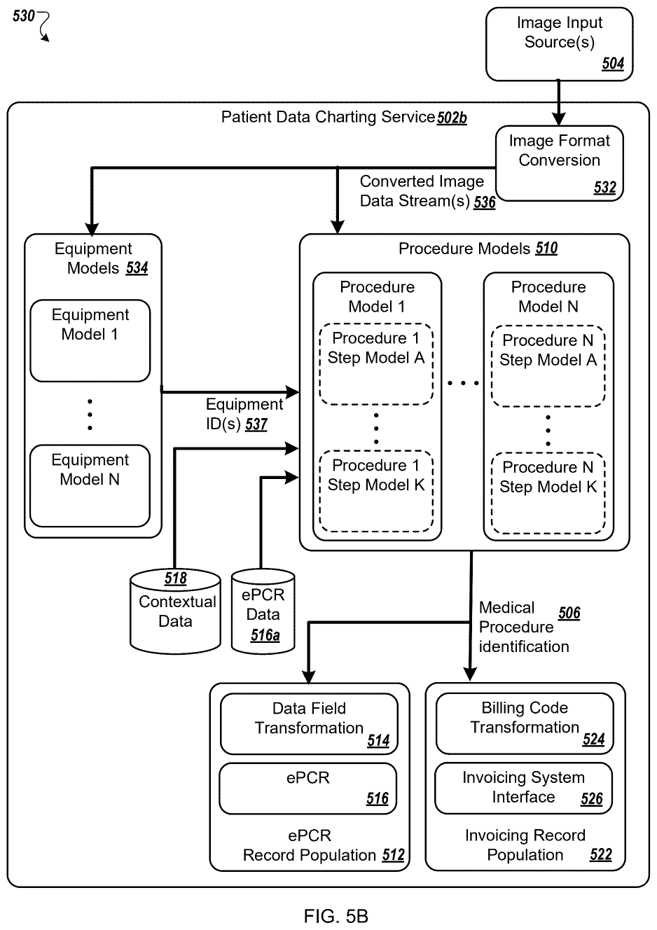

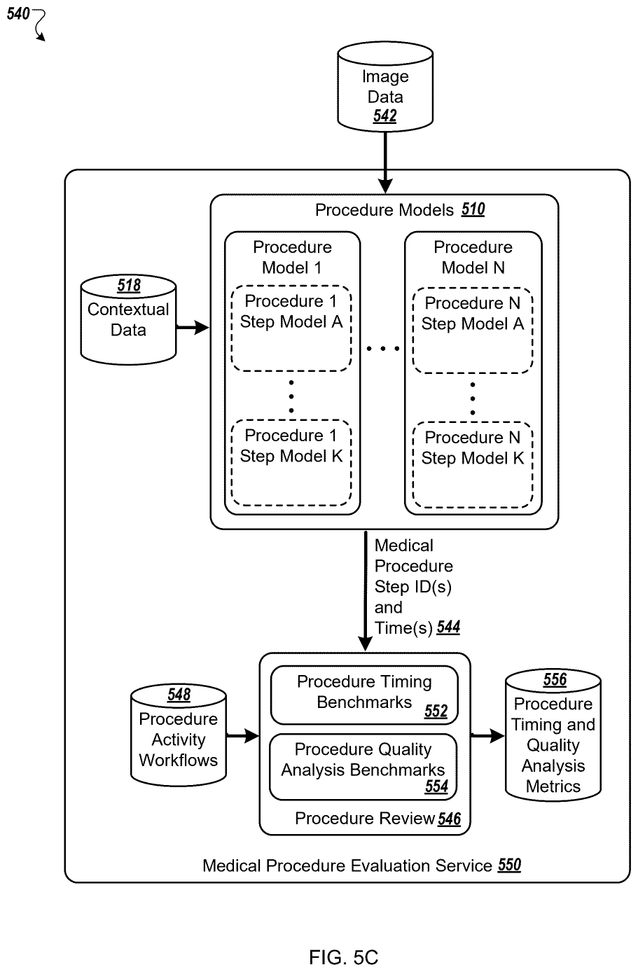

FIG. 5A through FIG. 5C are schematic illustrations of examples of emergency medical procedure identification and usage in the functioning of varying implementations of a patient data charting service 502. The patient data charting service 502 provides trained emergency medical procedure recognition models 510 for recognizing medical procedures in accordance with a series of steps of each medical procedure and applies the models 510 to a variety of uses. The patient data charting service 502, for example, may include portions of the patient data charting system 302 of FIG. 3. The patient data charting service 502 may provide the patient charting application 116 of FIG. 1B and/or the patient charting application 406a-c of FIG. 4A and FIG. 4B.

Turning to a first example emergency medical procedure identification and usage scenario 500 of FIG. 5A, in some implementations, data collected by one or more image input sources 504 are provided to the procedure models 510 of a patient data charting service 502a. As illustrated, a collection of N procedure models 510 are included in the collection, each procedure model relating to a medical procedure having a series of A through K steps. A total number of steps of each procedure model 510 may be vary. The procedure models 510, for example, may be the medical procedure models 356 of FIG. 3. Each procedure model 510 may be configured to recognize a given medical procedure of a collection of medical procedures (e.g., as described in relation to the precursor step identification engine 318, the procedure monitoring engine 320, and/or the activity identification engine 332 of FIG. 3).

The procedure models 510, in some embodiments, form part of an artificial intelligence (AI) analytics processor systems, where each procedure model 510 may be considered a separate AI model corresponding to a given procedure of a collection of medical procedures. The procedure models 510, thus, may each be configured to process image data in view of a corresponding medical procedure based on the training applied to the particular procedure model 510.

In some implementations, the procedure models 510 obtain contextual data 518 for use in analyzing the image recordings provided by the image input source(s) 504. The contextual data 518, for example, may be gathered by the contextual information recognition engine 322 as described in relation to FIG. 3. The contextual data, in some embodiments, includes a portion of an ePCR data record 516 (e.g., ePCR data related to the medical emergency and already logged to an ePCR record population engine 512).

In some implementations, the procedure models 510 output a prediction of a medical procedure identification 506. The medical procedure identification 506, for example, can identify a medical procedure performed by caregivers such as an emergency medical technician (EMT) 520 as well as, in some embodiments, context information such as medical equipment used, identification of a caregiver performing the medical procedure, an indication of success or failure of the medical procedure, and/or a skill level or accreditation level associated with the caregiver performing the medical procedure.

The predicted medical procedure identification 506, in some implementations, is presented to a caregiver (e.g., the EMT 520) via a user interface 508. The user interface 508, in some embodiments, prompts the EMT 520 for confirmation of correctness of the information. In some embodiments, the user interface 508 updates the ePCR data presentation based on additional information added to the ePCR data record 516. The user interface 508, for example, may present the information graphically and/or audibly. In one example, the user interface 508 may be presented as described in relation to the patient data charting GUI engine 338.

The confirmation and/or correction(s) provided by the EMT 520, in some implementations, are provided as confirmed medical procedure identifier(s) 519 for logging with the ePCR record population engine 512. The ePCR record population engine 512, for example, may perform operations described in relation to the ePCR coding conversion engine 336 of FIG. 3.

In some implementations, whether or not a confirmation process is conducted beforehand, the procedure identifiers (e.g., the predicted medical procedure identification 506 and/or the confirmed medical procedure identifier(s)) 519 are provided to the ePCR record population engine 512 for translating into ePCR record fields using a data field transformation architecture 514. The data field transformation architecture 514 may take the form of one or more search trees, data look-up tables, and/or other mapping data structures for mapping the medical procedure identification 506 to field data of the ePCR record 516. The translation, for example, may be performed as described in relation to the ePCR coding conversion engine 112 of FIG. 1B.

In some implementations, the medical procedure identification 506 is accessed by an invoicing record population engine 522 for translating into billing codes using a billing code transformation architecture 524. The billing code transformation architecture 524 may take the form of one or more search trees, data look-up tables, and/or other mapping data structures for mapping the medical procedure identification 506 to one or more billing codes. The translation, for example, may be performed as described in relation to the billing category conversion engine 134 of FIG. 1C. The invoicing record population engine 522 may transmit the billing codes to an invoicing system, such as the billing system 308 of FIG. 3, via an invoicing system interface 526. The invoicing system interface 526, for example, may include one or more APIs, direct communication channels, server transfers (e.g., file server transfers), shared storage regions, or other transmission mechanisms for transferring the billing codes to a billing system external to the patient data charting service.

In a second example emergency medical procedure identification and usage scenario 530 of FIG. 5B, the procedure models 510, ePCR record population engine 512, and invoicing record population engine 522 remain part of a patient data charting service 502b. In addition, the patient data charting service 502b includes an image format conversion engine 532 (e.g., the image format conversion engine 324 of FIG. 3) and a set of equipment models 534 (e.g., the medical equipment models 369 of FIG. 3).

In some implementations, the image format conversion engine 532 receives image recordings for the image input source(s) 504 and converts at least a portion of the image recordings to one or more converted image data streams 536. The converted image data stream(s) 536, in turn, are provided to both the procedure models 510 and the equipment models 534. In certain embodiments, portions of the converted image data stream(s) 536 may be provided to each of the procedure models 510 and the equipment models 534. For example, a different type of image data may be more appropriate to the equipment models 534 than to the procedure models 510. Additionally, the converted image data stream(s) 536 may not be provided concurrently to both of the procedure models 510 and the equipment models 534. In some embodiments, the equipment models 534 receive and analyze the converted image data stream(s) 536 prior to recognition of the beginning of a medical procedure, for example for inventory purposes, as described in relation to the medical equipment recognition engine 330 of FIG. 3.

In some implementations, the image format conversion engine 532 combines or co-registers data recordings from two or more image input sources 604 to enhance the analysis of the image data. For example, certain procedure models 510 and/or equipment models 534 may be configured to analyze co-registered image data (e.g., camera data and LiDAR data) to better recognize objects in the image recordings.

The image format conversion engine 532, in some implementations, adjusts the format of the image recordings from one or more image input sources 504 to an input compatible with certain procedure models 510 and/or equipment models 534. For example, the image format conversion engine 532 may reduce a number of colors in still or video camera data, reduce a resolution of (e.g., compress) an image recording, convert LiDAR recordings from multiple LiDAR sensors to three-dimensional LiDAR point cloud data, convert LiDAR recordings to wireframe format, and/or convert camera recordings from multiple cameras to three-dimensional camera data.

In some implementations, the image format conversion engine 532 performs conversion in part based on available processing circuitry and/or memory to perform image analysis with the procedure models 510 and/or equipment models 534. For example, if executing locally on a tablet or laptop computer, a reduced color and/or reduced resolution format of the image recordings may correlate to procedure models 510 and/or equipment models 534 requiring fewer processing cycles and/or less memory for performing the medical procedure identification 506. Conversely, rich models accepting three-dimensional LiDAR or camera data may be executed when adequate processing cycles and/or memory is available (e.g., upon network availability and/or edge server availability).

In some implementations, the equipment models 534 supply one or more equipment identifiers 537 for use by the procedure models 510 as context data (e.g., in addition to the contextual data 518 and the portion of the ePCR data 516a). The equipment identifiers 537, for example, may be the medical equipment identifiers 350 of FIG. 3.

In a third example emergency medical procedure identification and usage scenario 540 of FIG. 5C, a medical procedure evaluation service 550, which may be part of the patient data charting service 502 or may be a separate service, accesses image data 542 (e.g., collected by the one or more image sources 504 of FIG. 5A and FIG. 5B) for medical procedure performance analysis using the procedure models 510 in view of the contextual data 518. The image data 542 may be streamed data (e.g., in real-time or near real-time) or may be archived data (e.g., as described in relation to the image data archiving engine 344 of FIG. 3).

In some implementations, a portion of the procedure models 510 used by the medical procedure evaluation service 550 include contextually-enhanced procedure models configured to determine medical procedure step identifiers and step 544 of a medical procedure captured by the image data 542. Further, as described in relation to FIG. 1D, the portion of the procedure models 510 may be configured to determine timings (e.g., similar to the step identifiers and timings 158 of FIG. 1D) associated with each identified step of the medical procedure.

In some implementations, a procedure review engine 546 evaluates the series of medical procedure step identifiers and timings 544 in view of a procedure activity workflow of a collection of procedure activity workflows 548 corresponding to the medical procedure to evaluate procedure timing metrics in view of procedure timing benchmarks 552 and/or procedure quality analysis metrics in view of procedure quality analysis benchmarks 554. The procedure review engine 546, for example, may perform analysis similar to that described in relation to the procedure equality analysis engine 160 of FIG. 1D and/or the procedure quality analysis engine 342 of FIG. 3.

In some implementations, the procedure review engine 546 stores the procedure timing metrics and/or the procedure quality analysis metrics to a data store 556 for later evaluation. The procedure timing metrics and/or the procedure quality analysis metrics, for example, may be stored in relation to a caregiver performing the medical procedure to evaluate, in view of other quality-analyzed medical procedures performed by the caregiver, an ongoing performance record of the caregiver.

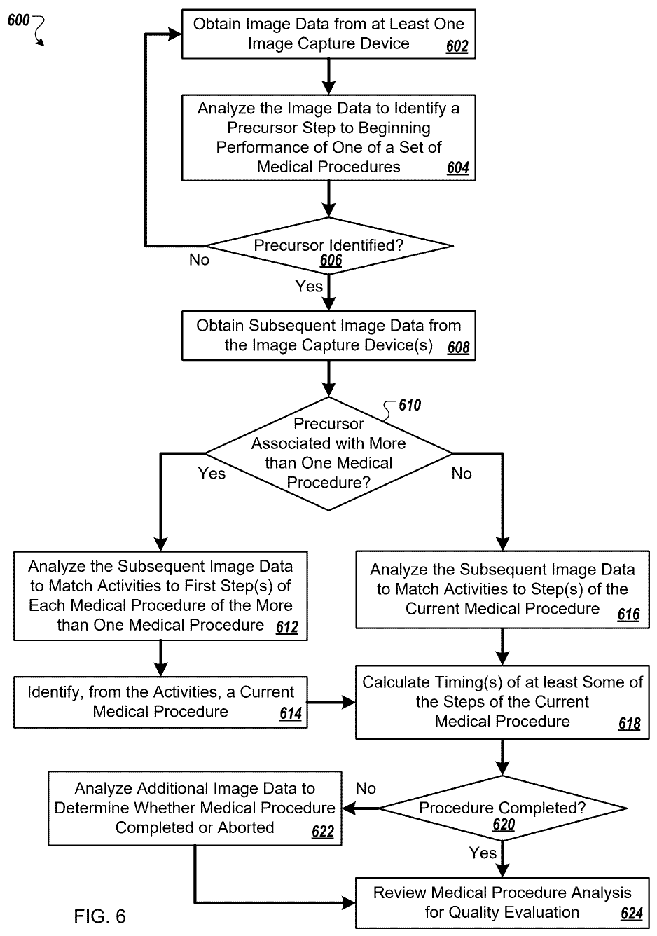

FIG. 6 is a flow chart of an example method 600 for analyzing image data to identify an emergency medical procedure. The method 600 may be performed, for example, by the patient charting system 302 of FIG. 3. Portions of the method 600, for example, may be performed by the patient data charting service 502 of FIG. 5A through FIG. 5C.

In some implementations, the method 600 begins with obtaining image data from at least one image capture device (602). The image data, for example, may be obtained by the image data collection engine 316 of FIG. 3. The image data may be accessed from a storage region, such as the image data 382 of FIG. 3 and/or the image data 542 of FIG. 5C. The image data, in some embodiments, is converted image data that has been combined, co-registered, and/or modified in format, as described in relation to the image format conversion engine 324 of FIG. 3 and/or the image format conversion engine 532 of FIG. 5B.

In some implementations, the image data is analyzed to identify a precursor step to beginning performance of one of a set of medical procedures (604). For example, the precursor step may be identified as described in relation to the precursor step identification engine 152 of FIG. 1D and/or the precursor step identification engine 318 of FIG. 3. In illustration, the precursor step(s) for a cricothyrotomy may include cleaning the area of the patient with an alcohol swab and palpitating with the cricothyroid membrane for landmarks.

In some implementations, while a precursor is not identified (606), subsequent image data is obtained (602) and analyzed (604). The method 600, for example, may continue to monitor image data for evidence of a precursor step to the beginning of a medical procedure.

Once a precursor step has been identified (606), in some implementations, subsequent image data is obtained from the image capture device(s) (608). The subsequent image data may include image data from the same image source(s) and/or image data from additional image source(s). Further, in some embodiments, the subsequent image data obtained may have been converted to a different format than the image format(s) used for identifying the precursor step. In illustration, depending upon formats compatible with one or more models for identifying a cricothyrotomy procedure, the compatible image format(s) may be obtained. Further, based on the vantage point required to monitor performance of a cricothyrotomy procedure, one or more image sources capturing the target region may be the focus of further image data for medical procedure performance analysis.

In some implementations, if the precursor step is associated with more than one medical procedure (610), the subsequent image data is analyzed to match activities to one or more initial steps of each medical procedure of the multiple medical procedures (612). For example, the procedure monitoring engine 320 of FIG. 3 may analyze the subsequent image data using medical procedure models 356 corresponding to each medical procedure of the potential medical procedures that the caregiver(s) is preparing to perform.

In some implementations, a current medical procedure is identified from the activities captured in the subsequent medical data (614). For example, after identifying one or more further steps by analyzing the subsequent image data in view of medical procedure models trained to recognize each potential medical procedure, the multiple procedures may be narrowed down to a particular medical procedure.

In some implementations, timings of at least some of the steps of the current medical procedure are calculated (618). The timings, for example, may be calculated as described in relation to the procedure timing engine 334 of FIG. 3.

In some implementations, if the medical procedure is deemed to have not been completed (620), additional image data is analyzed to determine whether the medical procedure is eventually completed or has been aborted (622). During the medical procedure, setbacks may occur, and some steps may need to be repeated to complete the procedure. Conversely, in some circumstances, the medical procedure may need to be aborted as a failure. When the series of activities performed by the caregiver(s) fail to generally follow the procedure activity workflows 352 of FIG. 3 or other medical procedure protocol, in some embodiments, the method 600 revisits the subsequent image data to recognize activities that correspond to repetitions and/or aborting the medical procedure.

In some implementations, if the medical procedure is determined to have been completed (620), the medical procedure analysis is reviewed for quality evaluation (624). Determining completion of the medical procedure, for example, may include identifying a completion step of one or more completion steps corresponding to the medical procedure (e.g., according to a medical procedure workflow or protocol). In some embodiments, whether successful or aborted, the medical procedure may be analyzed to determine quality metrics and/or ratings corresponding to the caregiver(s)'s performance of the medical procedure. For example, the medical procedure performance may be evaluated as described in relation to the procedure quality analysis engine 160 of FIG. 1D and/or the procedure quality analysis engine 342 of FIG. 3.

Although illustrated as a certain series of operations, in other embodiments, the method 600 may include more or fewer operations. For example, while identifying the current medical procedure (614), timings of each overlapping step between multiple potential medical procedures may be timed (618). In further embodiments, portions of the operations of the method 600 may be performed in a different order and/or concurrently. For example, timings may be calculated concurrently with analyzing the subsequent image data to match the activities to steps of the current medical procedure (616). Other modifications of the method 600 are possible while remaining within the spirit and scope of the disclosure.

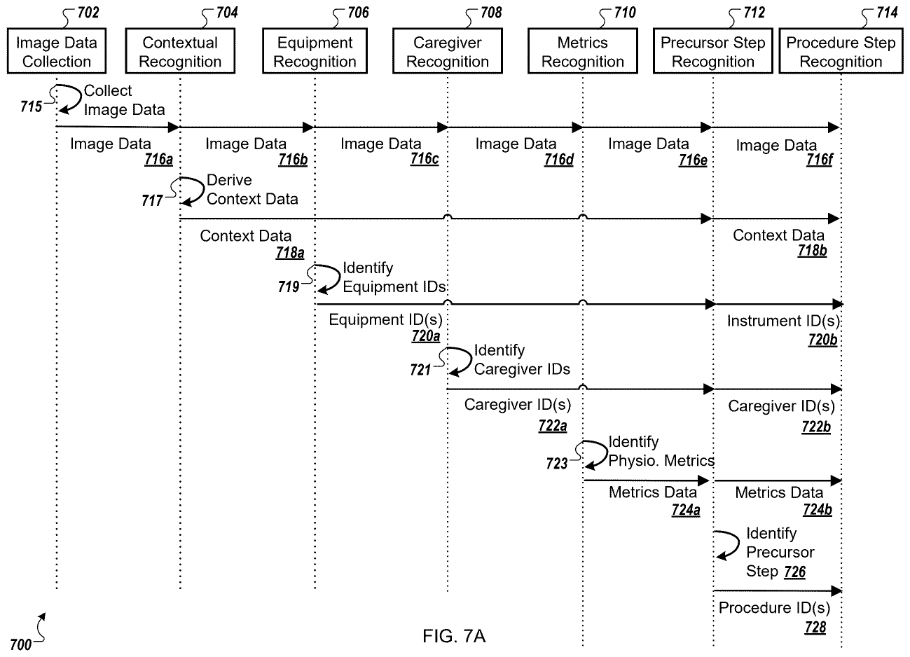

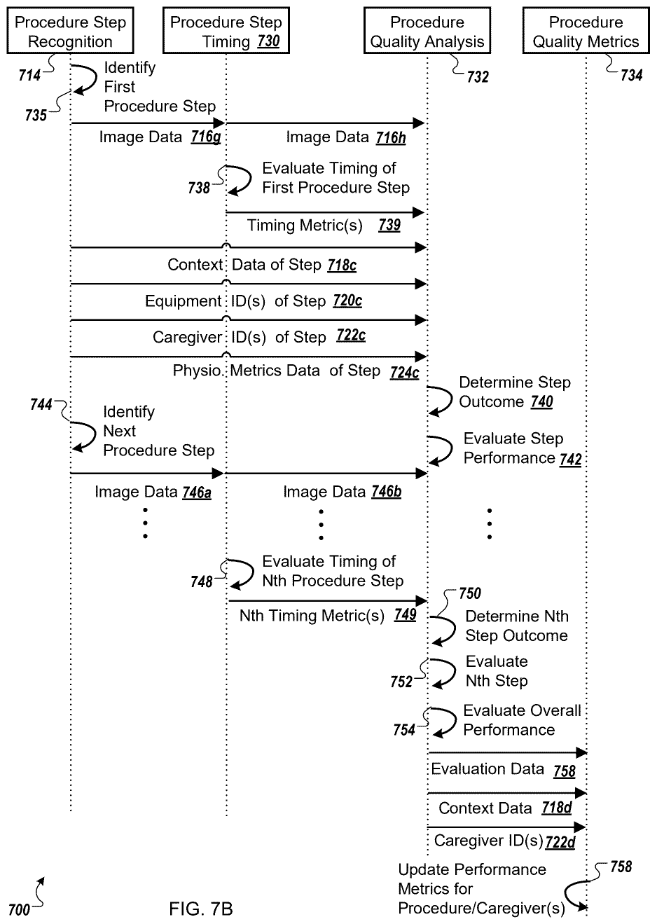

FIG. 7A and FIG. 7B are swim lane diagrams of an example method 700 for analyzing image data to identify an emergency medical procedure and automatically evaluate the performance of the procedure. The method involves a collection of services working in collaboration to conduct an objective evaluation of the medical procedure captured in the image data. The services, in some examples, may include individual algorithms, software modules, and/or microservices designed to interoperate in a pipelined fashion to gather information needed for the evaluation and to analyze the information to determine performance metrics. Although illustrated as a particular pipelining architecture, the architecture presented is illustrated for ease of demonstration and does not necessarily reflect the architecture of the communications between the various services of the method 700. The method 700, for example, may be performed by the patient charting system 302 of FIG. 3 or the patient data charting service 502c of FIG. 5C.

In some implementations, the method 700 begins with collecting (715), by an image data collection service, image data for use during performance of the method 700. The image data collection service, for example, may perform certain operations described in relation to the image capture control engine 314 and/or the image data collection engine 316 of FIG. 3. The image data, for example, may correspond to one or more image recordings captured by one or more image capture devices, such as the image capture devices 304 of FIG. 3.

In some implementations, the image data is obtained (716a) by a contextual recognition service 704. The contextual recognition service 704, in some implementations, derives (717) context data related to (e.g., within a same timeframe or a preceding timeframe of) the image data. The contextual recognition service 704, for example, may perform operations described in relation to the contextual information recognition engine 322 of FIG. 3. The context data, in some examples, may include the dispatch information 364, the medical device metrics 366, and/or the physiological data 360 collected from other devices at the emergency medical scene. Further, the context data may include already logged ePCR data 380. Although illustrated as obtaining the image data itself, the context recognition service 704, due to not necessarily needing to analyze the image data to derive the context data, may instead be instructed to collect context data from a relevant timeframe to the image data. In other embodiments, the context recognition service 704 may obtain the image data to distribute the image data to other services designed to derive additional context data from the image data itself, as follows.

In some implementations, the context data is obtained (718a) by a precursor step recognition service 712. The precursor step recognition service 712 also obtains (716f) the image data.

In some implementations, the image data is obtained (716b) by an equipment recognition service 706 (e.g., from the image data collection service 702 or the contextual recognition service 704). The contextual recognition service 704, for example, may provide the image data to the equipment recognition service 706 absent sufficient medical device metrics obtained from data supplied by medical devices at the emergency medical scene. The equipment recognition service 706, for example, may be configured to apply the medical equipment models 368 of FIG. 3 to recognize medical equipment in the image data as described in relation to the medical equipment recognition engine 330. In some embodiments, the equipment recognition service 706 identifies (719) medical equipment items as one or more equipment identifiers.

In some implementations, the equipment identifiers are obtained (720a) by the precursor step recognition service 712.

In some implementations, a caregiver recognition service 708 obtains (716c) the image data (e.g., from the image data collection service 702 or the contextual recognition service 704) and identifies (721) one or more caregivers performing the medical procedure by one or more caregiver identifiers. The contextual recognition service 704, for example, may provide the image data to the caregiver recognition service 708 absent sufficient caregiver information obtained from the dispatch information 364. The caregiver recognition service 708, for example, may perform operations described in relation to the caregiver identification engine 326 of FIG. 3 to determine the caregiver identifiers 362.

In some implementations, the caregiver identifiers are obtained (722b) by the precursor step recognition engine 712.

In some implementations, a metrics recognition service 710 obtains (716d) the image data (e.g., from the image data collection service 702 or the contextual recognition service 704) and identifies (723) physiological metrics from the image data. The contextual recognition service 704, for example, may provide the image data to the metrics recognition service 710 absent sufficient physiological data obtained from the medical device metrics 366. The metrics recognition service 710, for example, may perform operations described in relation to the physiological metrics recognition engine 328 of FIG. 3.

In some implementations, the physiological metrics data is obtained (724a) by the precursor step recognition service 712.

In some implementations, the precursor step recognition service 712 identifies (726), from the image data, one or more procedure identifiers corresponding to one or more precursor steps (e.g., one or more activities performed by caregivers in preparation for performing a medical procedure). The precursor step recognition service 712, for example, may apply the context data, the equipment identifier(s), the caregiver identifier(s), and/or the physiological metrics data in determining which medical procedure(s) may correspond to the precursor step(s) identified in the image data. The precursor step recognition service 712, for example, may perform operations described in relation to the precursor step identification engine 318 of FIG. 3.

In some implementations, a procedure step recognition service 714 obtains the image data (716f) and the procedure identifier(s) (728). The image data, in this circumstance, includes image data of activities following the precursor step(s) performed by the caregiver(s). The procedure step recognition service 714 may also obtain one or more of the contextual data (718b), the instrument identifier(s) (720b), the caregiver identifier(s) (722b), and/or the metrics data 724b. Although illustrated as being the same data, in some embodiments, one or more of the contextual data (718b), the instrument identifier(s) (720b), the caregiver identifier(s) (722b), or the metrics data 724b may be updated to capture and/or include additional information identified in image data collected subsequent to performance of the precursor step(s).

Turning to FIG. 7B, in some implementations, the procedure step recognition service 714 identifies (735) at least a first medical procedure step following the precursor step(s) using the image data as well as, in some embodiments, the contextual data (718b), the instrument identifier(s) (720b), the caregiver identifier(s) (722b), and/or the metrics data 724b. The procedure step recognition service 714 may also include timing information, such as a begin time and an end time (or an elapsed time) corresponding to the first procedure step. The procedure step recognition service 714, for example, may perform operations as described in relation to the procedure monitoring engine 320 of FIG. 3.

In some implementations, a procedure step timing service 730 obtains (716g) the image data (e.g., from the procedure step recognition service 714 or the image data collection service 702). The procedure step timing service 730, in some embodiments, also obtains (739) one or more timing indicators (e.g., beginning and end times, an elapsed time, etc.) from the procedure step recognition engine 714.

In some implementations, the procedure step timing service 730 evaluates (738) a length of time of the first procedure step. The procedure step timing service 730, for example, may perform a portion of the operations described in relation to the procedure quality analysis engine 342 of FIG. 3. For example, the procedure step timing service 730 may apply the procedure timing benchmarks 552 of FIG. 5C to calculate procedure timing metrics.

In some implementations, a procedure quality analysis service 732 obtains (716h) the image data corresponding to at least the first step (e.g., from the procedure step recognition service 714 or the image data collection service 702). In some implementations, the procedure quality analysis service 732 obtains (739) one or more timing metrics from the procedure step timing service 730. Further, the procedure quality analysis service 732 may obtain contextual data corresponding to the step (718c), equipment identifier(s) corresponding to the step (720c), caregiver identifier(s) corresponding to the step (722c) and/or physiological metrics data corresponding to the step (724c).

In some implementations, the procedure quality analysis service 732 determines (740) a step outcome based at least in part on the timing metric(s) 739 as well as, in some embodiments, one or more of the image data, the context data, the equipment identifier(s) the caregiver identifier(s) and/or the physiological metrics data. The step outcome, for example, may represent success and/or failure of the particular step. Further, the step outcome may represent adherence, in performance of the step, to a medical procedure protocol corresponding to the medical procedure. The outcome, in some embodiments, is determined only for particular steps, such as a final step of the medical procedure. The procedure quality analysis service 732, for example, may perform a portion of the operations described in relation to the procedure quality analysis engine 342 of FIG. 3. For example, the procedure quality analysis service 732 may apply the procedure quality analysis benchmarks 554 of FIG. 5C to calculate procedure quality analysis metrics.

In some implementations, the procedure quality analysis service 732 evaluates (742) step performance based at least in part on the timing metric(s) 739 as well as, in some embodiments, one or more of the image data, the context data, the equipment identifier(s) the caregiver identifier(s) and/or the physiological metrics data. The step performance, in some examples, may represent analysis of the image data representing handling of performing a given step, as described, for example, in relation to the procedure quality analysis engine 342 of FIG. 3 and/or the procedure quality analysis benchmarks 554 of FIG. 5C.

In some implementations, for remaining steps of the medical procedure, the procedure step recognition service 714 identifies (744) the next procedure step, and the procedure step timing service 730 obtains (746a) subsequent image data corresponding to the next procedure step (in addition to, in some embodiments, additional context, equipment, caregiver, and/or physiological information) to evaluate (748) timing of the next procedure step.

In some implementations, the procedure step recognition service 714 is configured to recognize activities corresponding to a completion step associated with the medical procedure (e.g., in accordance with the medical procedure workflow and/or protocol). Some medical procedures may include more than one completion step, for example where two or more steps may be performed a different order. In absence of a completion step, the procedure step recognition service 714 may identify that the caregiver(s) have moved on from the medical procedure (e.g., abandoned or aborted the procedure).

Further, for the remaining steps of the medical procedure, in some implementations the procedure quality analysis service 732 obtains (746b) image data for the next procedure step, obtains (749) timing metrics from the procedure step timing service 730 as well as, in some examples, any other context, equipment, caregiver, and/or physiological information corresponding to the next step, to determine (750) the outcome of the Nth step and to evaluate (752) performance of the Nth step.

In some implementations, using the step outcome determinations and the step performance evaluations, the procedure quality analysis service 732 evaluates (754) the overall performance of the medical procedure. The step outcomes and step evaluations, for example, may be combined to produce an overall grade, rating, or other metric for benchmarking the performance against historic performance of the caregiver and/or other caregivers.

In some implementations, evaluation data produced by the procedure quality analysis service 732 are provided (758) to a procedure quality metrics service 734 for collection and/or aggregation with past metrics. The procedure quality analysis service 732 may also obtain, in some examples, the context data (718d) and/or the caregiver identifier(s) (722d) associated with performance of the medical procedure. For example, the procedure quality analysis service 732 may update (758) performance metrics for one or more caregivers identified with the caregiver identifier(s). Further, the procedure quality analysis service 732 may update (758) performance metrics related to general performance of the procedure. The context data, in some examples, may be used to gather metrics related to a particular emergency transport vehicle company, a particular emergency transport vehicle, a particular geographic region, a particular caregiver team, and/or a particular level of training/accreditation/skill level of caregiver.

FIG. 8A through FIG. 8C are block flow diagrams of example processes for training machine learning models, such as the medical procedure models 356 of FIG. 3, to analyze image data to identify emergency medical procedures. Each training example of FIG. 8A through FIG. 8B may obtain differing input data to derive procedure models dedicated to recognizing medical procedures for different purposes, such as ePCR field data population, invoicing, and quality assurance evaluations.

Turning to FIG. 8A, in some implementations, an example process 800 for training and updating procedure models for identifying one of a collection of medical procedures begins with building a set of truth data 802 with labeled images. The truth data 802 may include labels within and/or correlated to each image data file. In a first example, the labels may include image markers (e.g., scene breaks) and/or logical markers (e.g., timestamps, bitmap coordinates, wireframe coordinates, etc.) identifying a beginning of each step of a series of steps of a given medical procedure. In a second example, the labels may include digital labels (e.g., overlaid on one or more still items or video frames) and/or other object identifiers (e.g., physical tags/stickers applied to medical equipment) for uniquely identifying various medical equipment items used during the medical procedure. Further, the truth data may include one or more physical or logical labels identifying conclusion of the medical procedure (e.g., scene break and/or timestamp).

The truth data 802, in some implementations, includes multiple types of image data such as, in some examples, full color video image data, black and white video image data, full color still image series, black and white still image series, wireframe LiDAR data, point cloud LiDAR data, three-dimensional bitmap data, and/or three-dimensional point cloud LiDAR data. The labeling used in the truth data may differ based on a type of data. For example, colorful labels will not work well in black and white video data, and visual shape labels will not work well in wireframe data. In some embodiments, a portion of the image data capturing a particular performance of a medical procedure as recorded by multiple image capture devices may be co-registered for combined analysis. In this manner, for example, the labels applied to one set of data may be mapped to the corresponding image data captured by another image capture device.

In some implementations, a procedure model training engine 804 analyzes the truth data 802 corresponding to each medical procedure of the collection of medical procedures to generate one or more sets of trained models 806a (e.g., at least one trained model per medical procedure, at least one trained model per medical procedure per image data type, etc.). The procedure model training engine 804, for example, may perform operations of the procedure model training engine 346 of FIG. 3 to produce the medical procedure models 356.

In some embodiments, at least a portion of the trained models 806a include one or more deep neural network (DNN) models each configured to identify a given medical procedure based on a series of steps (e.g., actions) associated with the given medical procedure, as well as one or more medical equipment items used in performance of the given medical procedure. DNN models, for example, perform regression and classification on data input. In some embodiments, at least a portion of the trained models 806a include one or more convolution neural network (CNN) models. CNN models, for example, are designed to break down features of an image into sub-features, making CNN classification particularly advantageous for image classification. In some embodiments, at least a portion of the trained models 806a include one or more network in network (NiN) models. NiN models, for example, take CNN processing to another level by analyzing a network of convolutional layers of an image, proving advantageous for image classification. Other deep learning models may be applied to training the trained models 806a, with the particular deep learning model being selected, in some examples, based in part on processing availability and storage size availability in the end system (e.g., a cloud network versus processing on a tablet computing device), third party tool access (e.g., availability of cloud provider specialized tools and hardware for performing image classification), and/or processor type (e.g., GPU, CPU, field-programmable gate array (FPGA), etc.).

In some implementations, the trained models 806a are stored to a trained procedure model data store 808. The data store 808, for example, may be a network-accessible (e.g., cloud or server farm) storage region communicatively connectable to various patient charting systems for executing the trained procedure models 806a and/or for downloading a portion of the trained procedure models 806a for local execution (e.g., on one or more computing devices located at an emergency medical scene).

In some implementations, at a later time, one or more trained models 806b (e.g., a subset of the trained models 806a, such as models configured to recognize a particular medical procedure or set of related (e.g., similar) medical procedure, may undergo further training by a procedure model updating engine 812. The procedure model updating engine 812, for example, may access field-captured image data 810 (e.g., such as image data archived by the image data archiving engine 344) for refining the training of the trained models 806b. The procedure model updating engine 812 may be configured to update the trained models 806b in conformance with the one or more types of machine learning models and/or classifiers used to produce the trained models 806a (e.g., DNN, CNN, NiN, etc.). The field-captured image data 810 may include limited or no truth labeling.

In some embodiments, the field-captured image data 810 is confirmed to represent a particular medical procedure. For example, one or more procedure identifiers 110 applied to the image data through analysis (e.g., by the activity identification engine 106 of FIG. 1B and FIG. 1B, the procedure monitoring engine 156 of FIG. 1D, the procedure monitoring engine 320 of FIG. 3 and/or the activity identification engine 332 of FIG. 3) may be cross-referenced with confirmed data regarding performance of the medical procedure. The confirmed data, in some examples, may include confirmed invoicing data and/or ePCR data related to the emergency medical scene at which the field-captured image data 810 was recorded. In some embodiments, if confirmation results in a failure to match the one or more procedure identifiers 110 to confirmed data, the field-captured image data 810 may be flagged as negative training data (e.g., an example of performance of some other procedure that is not the medical procedure).

In some embodiments, a set of one or more updated trained models 814 produced by the procedure model updating engine 812 are stored to the trained procedure model data store 808. The updated trained model(s) 814 may replace the trained models 806b or be stored as newer version(s) of the trained model(s) 806b.

Turning to FIG. 8B, an example process 820 for training context-aware procedure models for identifying one of a collection of medical procedures, in some implementations, begins with obtaining image data 822 as well as context data 824. The image data may be truth data, such as the truth data 802 of FIG. 8A and/or field-captured image data such as the field-captured image data 810 of FIG. 8A. The context data 824 may include any or all of the types of context data discussed in relation to FIG. 7A such as, in some examples, the ePCR data 380, the dispatch information 364, the medical equipment identifiers 350, the caregiver identifiers 362, the physiological data 360, and/or the medical device metrics 366 of FIG. 3. The context data 824 and the image data 822, for example, may have been archived by the image data archiving engine 344 of FIG. 3.

In some implementations, the procedure model training engine 804 trains one or more trained context-aware procedure models 826 using the image data 822 and associated context data 824. The training, for example, may be performed as described in relation to FIG. 8A.

In some implementations, the procedure model training engine 804 also trains the trained context-aware models 826 using a set of trained models 806c (e.g., at least a portion of the trained models 806a and/or the trained models 806b). In this manner, the procedure model training engine 804 may behave similar to the procedure model updating engine 812 in that it refines the trained models 806c in view of the new data 822 and 824. However, the procedure model training engine 804, in introducing the context data 824 into the training set, may reduce a footprint of the trained context-aware models 826 as compared to the trained models 806c. For example, the machine learning classifiers produced using the associated context data may be smaller than a corresponding trained model 806a or 806b, such that a memory space and computation requirements of the trained context-aware models 826 are less than half the required memory space and/or processing resources required to execute their corresponding trained procedure models 806a or 806b.

In some implementations, the trained context-aware models 826 are stored to a trained context-aware procedure model data store 828. The data store 828 may be maintained together (e.g., in a shared storage region) as the trained procedure models of the trained procedure model data store 808 or in a separate location.

Turning to FIG. 8C, an example process 830 for training procedure quality analysis models for identifying discrete steps of one of a collection of medical procedures, in some implementations, begins with obtaining image data 832 as well as quality analysis truth data 834. The quality analysis truth data 834, as described in relation to FIG. 8A, may include labels correlated to each image data file of the image data 832. In a first example, the labels may include timestamps identifying a beginning of each step of a series of steps of a given medical procedure. Further, the quality analysis truth data 834 may include one or more logical labels identifying conclusion of the medical procedure (e.g., scene break and/or timestamp).

The quality analysis truth data 834, in some embodiments, includes negative truth data, such as labels identifying one or more failed steps, repeated steps, and/or an aborted procedure. The negative truth data may assist in recognizing imperfect performances of medical procedures and/or medical procedure performances that fail to fully comply with protocol and/or fail to follow the general workflow for the medical procedure.

In some embodiments, rather than or in addition to the quality analysis truth data 834, the image data 832 may be digitally labeled with truth data, for example as described in relation to FIG. 8A.

In some implementations, the procedure model training engine 804 accesses procedure workflows 836 and/or procedure protocols for matching identified steps of medical procedures with the anticipated series of steps. The procedure workflows 836, for example, may include decision tree models representing potential paths of steps to follow to perform a given medical procedure.

The procedure model training engine 804, in some implementations, trains a set of trained quality assurance models 838 using the image data 832 and at least one of the quality analysis truth data 834 or the procedure workflows 836. The procedure model training engine 804 may train the models, for example, in a manner such as that described in relation to FIG. 8A.

In some implementations, the trained quality assurance models 838 are stored to a trained procedure quality assurance model data store 840. The models of the data store 840 may be maintained together (e.g., in a shared storage region) with the trained procedure models of the trained procedure model data store 808 and/or the models of the trained context-aware procedure model store 828 or in a separate location. For example, since quality assurance analysis may oftentimes be performed on archived data rather than in real-time during a medical emergency, the trained procedure quality assurance model data store 840 may be maintained at a separate network connection.

FIG. 8D illustrates an example process 850 for training medical equipment recognition models for identifying medical equipment items used in each of a collection of medical procedures. The example process 850, for example, may be performed by the equipment recognition training engine 348 of FIG. 3. In some implementations, the example process 850 begins with obtaining, by the procedure model training engine 804, image data 852 as well as medical equipment truth data 854. The medical equipment truth data 854, for example, may include logical markers (e.g., bitmap coordinates, wireframe coordinates, etc.) and corresponding timestamps identifying various medical equipment items in the image data 852.

In some embodiments, rather than or in addition to the medical equipment truth data 854, the image data 852 may include captured truth data. The image data 852, for example, may include digital labels (e.g., overlaid on one or more still items or video frames) and/or other object identifiers (e.g., physical tags/stickers applied to medical equipment) for uniquely identifying various medical equipment items used during the medical procedure.

In some implementations, the procedure model training engine 804 obtains equipment context data 856. The equipment context data may include medical equipment identifiers recognized as being part of the emergency medical scene at which the image data 852 was captured based on, in some examples, an energy signature of a particular medical device, proximity data derived from near-field wireless communications or short-range wireless communications providing the identity and/or proximity of the medical device, and/or communication signals or signatures including a device identifier or other data individually identifying a medical equipment item and/or the type of that equipment. The equipment context data 856, further, may include medical equipment item usage derived from confirmed ePCR data and/or invoicing data from the emergency medical scene.

In some implementations, the procedure model training engine 804 trains a set of trained equipment models 858 using the image data 852. The procedure model training engine 804 may further train at least a portion of the trained equipment models 858 and at least one of the medical equipment truth data 854 or the equipment context data 856. The procedure model training engine 804 may train the models, for example, in a manner such as that described in relation to FIG. 8A.

In some implementations, the trained equipment models 858 are stored to a trained equipment identification model data store 860. The data store 860 may be maintained together (e.g., in a shared storage region) as the trained procedure models 808 of FIG. 8A, the trained context-aware procedure models 828 of FIG. 8B, and/or the trained procedure QA model data store 840 of FIG. 8C or in a separate location. The trained equipment identification models 858, for example, may be used by the medical equipment recognition engine 330 of FIG. 3 to perform an inventory analysis of the medical scene and/or to track medical equipment item objects at the emergency medical scene for identifying a precursor step to performing a medical procedure (e.g., as applied by the precursor step identification engine 318).

FIG. 9 illustrates an example of a logical and physical architecture 900 of a patient charting service as part of an SaaS platform 926 executing in a cloud environment 902. In some implementations, a patient data charting application 910 executing on a user interface device 906 in a mobile emergency medical services (EMS) environment 904 communicatively couples to a charting system server 918 of the SaaS platform 926 to provide patient charting services to a mobile team, such as a team of an EMS transport vehicle. The user interface device 906, in some examples, may be a smart phone, tablet, or laptop computer. The user interface device 906 may be part of a computing system integrated into an EMS transport vehicle housing the mobile EMS environment 904. The transport vehicle, in some examples may be an ambulance, a fire engine, an EMS crew transport vehicle, and/or a helicopter.

In some implementations, the mobile EMS environment 904 includes an edge server 914 hosting a patient data charting application 920a. The patient data charting application 920a may include the same capabilities of the patient data charting application 910 of the user interface device 906 and/or additional capabilities. For example, the patient data charting application 920a may include more processing intensive features that the hardware architecture of the user interface device 906 is incapable of performing. The edge server 914, for example, can be used to move a portion of the computing capability traditionally shifted to a cloud computing environment into the mobile EMS environment 904 so that any computation intensive data processing and/or analytics required by the user interface device 906 can run accurately and efficiently. The edge server 914, for example, may be configured to communicate with the user interface device 906 directly or via a network. For instance, the edge server 914 can include a private wireless network interface, a public wireless network interface, and/or a wired interface through which the edge server 914 can communicate with the user interface device 906 (as well as, in some embodiments, the medical device(s) 932). In some embodiments, certain devices of the mobile EMS environment 904 may be configured to communicate indirectly with the edge server, for example via another local device.

The edge server 914, in some embodiments, is a computing device configured to execute processor intensive operations that are sometimes involved when executing machine learning processes, such as natural language processing operations and/or computer vision processing operations. The edge server 914 may include, for example, one or more GPUs that are capable of efficiently executing matrix operations as well as substantial cache or other high-speed memory to service the GPUs. The edge server 914 may be a standalone physical device. The edge server 914 may be incorporated into other computing equipment, such as a laptop computer, tablet computer, medical device, or other specialized computing device. Alternatively or additionally, the edge server 914 may be located within a carrying case for such computing equipment. The edge server 914, in a further example, may be incorporated into the communications and processing capabilities of a mobile unit such as a vehicle or drone, or may otherwise be located within the mobile unit.

In some embodiments, the edge server 914 is used to support the user interface device 906 in the absence of a connection with a patient data charting application 920b of the platform 926. For example, the patient data charting application 910 may be configured to offload processing-intensive operations to the patient data charting application 920b when a strong (e.g., reliable, fast, high-bandwidth, etc.) network connection is available for obtaining real-time results of such computations. Conversely, absent the strong network connection, the patient data charting application 910 may be configured to shift a portion of its processing to the patient data charting application 920a executing on the edge server 914. Further, the edge server 914 may be configured to communicate with the platform 906 via one or more public or private wireless network interfaces, for example to transition functionality back to the patient data charting application 920b, and/or to upload data generated while executing on behalf of the patient data charting application 910.

In some implementations, the patient data charting application 910 and/or the patient data charting application 920a interoperate with a positioning system 940 included in the mobile EMS environment 904. The positioning system 940 may use global positioning system data (e.g., GPS satellite positioning) and/or cellular positioning data to locate the user interface device 906. The patient charting application 910 and/or the patient data charting application 920a may use the positioning data to determine a context for the user interface device 906, and this determined context may enable the patient data charting application 910 and/or the patient data charting application 920a to select and adapt image data capture and/or analysis, for example as described in regard to FIG. 5B.

In some implementations, the patient data charting application 910 and/or the patient data charting application 920a receives and utilizes data from other elements of the SaaS platform 926 executing in the cloud environment 902. The platform 926 may include, in various embodiments, a CAD system server 930, a navigation system server 928, a medical billing system server 967, a medical device case data store 924, a charting system data store 920 and/or a patient data charting application 920b. In some implementations, the SaaS platform 926 enables sharing of information between entities of the platform and enables the patient data charting application 910 and/or the patient data charting application 920a to enhance patient care through offloading some of the charting efforts from the emergency caregivers, allowing the caregivers to focus on providing immediate care. For example, the patient data charting application 910 and/or the patient data charting application 920a may identify a medical procedure during and/or immediately after performance and log information identifying the procedure performed in the charting system data store 920. In another example, the patient data charting application 910 and/or the patient data charting application 920a may automatically identify an emergency medical procedure, classify the procedure as belonging to a certain billing category, and provide the billing category information to the medical billing system server 967. In a further example, the patient data charting application 910 and/or the patient data charting application 920a may automatically identify an emergency medical procedure and log details regarding the procedure, such as whether it was successful or unsuccessful, in the case data store 924.

The cloud environment 902 of FIG. 9, in some embodiments, may be implemented within a data center or other high-capacity computing facility with highspeed internet connectivity. For instance, the cloud environment 902 may be implemented via a commercially available cloud computing service, such as Microsoft® Azure, Google Cloud Platform™, or Amazon™ Web Services (AWS™). The platform 926 may include a number of dedicated servers (e.g., a farm or cluster of computing systems) within the data center that are interconnected via a high speed, private network. Each of the servers illustrated within the platform 926, for example, may be implemented by one or more physical and/or virtual servers. The servers can include one or more application servers, web servers, and/or database servers. The servers can include enterprise servers, configured to support an organization as a single tenant and/or cloud servers configured to support multiple organizations as multiple tenants.

The services (e.g., software applications, algorithms, and/or routines programmed to programmable processing circuitry) hosted by servers within the platform 926, in some embodiments, are configured to expose application programming interfaces (APIs) that enable the services to communicate with one another. These APIs, for example, may be configured to receive, process, and respond to commands issued by services hosted on the same server or a different server in the platform 926. For instance, these APIs may enable any of the servers of the platform 926 to transmit queries, information, patient reference codes, etc. and otherwise communicate with one or more other servers in the platform 926 and/or with the patient data charting application 910 and/or the patient data charting application 920a. The APIs may be implemented using a variety of interoperability standards and/or architectural styles. In one example, the APIs are web services interfaces implemented using a representational state transfer (REST) architectural style. In this example, the APIs communicate with a client process using Hypertext Transfer Protocol (HTTP) along with JavaScript Object Notation (JSON) and/or extensible markup language (XML). In some embodiments, portions of the HTTP communications are encrypted to increase security. Alternatively or additionally, in some implementations, the APIs are implemented as a .NET web API that responds to HTTP posts to particular uniform resource locators (URLs). Alternatively or additionally, in some embodiments, the APIs are implemented using simple file transfer protocol (SFTP) commands and/or a proprietary application protocol accessible via a transmission control protocol socket. Thus, the APIs described herein are not limited to a particular implementation.

The network architecture within the cloud environment 902 and the local network architecture within the mobile EMS environment 904 can include one or more communication networks through which the computing devices within these environments send, receive, and/or exchange data. In various embodiments, the network can include a cellular communications network and/or a computer network. In some implementations, the network architecture(s) includes and supports wireless network connections and/or wired connections. For instance, the network architecture(s) may support one or more networking standards including personal area network (PAN) standards, such as universal serial bus (USB), Bluetooth®, controller area network (CAN) standards, or Zigbee®; one or more local area network (LAN) standards such as wireless Ethernet, Ethernet, and/or transfer control protocol/internet protocol (TCP/IP); and one or more wide area network (WAN) standards such as, in some examples, TCP/IP, global system for mobile (GSM), and/or code-division multiple access (CDMA). As such, the network may include both private networks, such as LANs, and public networks, such as the Internet. In some embodiments, the network may include one or more intermediate devices involved in the routing of communications (e.g., packets) from one endpoint to another. However, in other embodiments, the network involves only two endpoints that each have a network connection directly with the other.

The charting system data store 920, in some embodiments, is implemented by a database (e.g., a relational database) and stored on one or more non-transitory (non-volatile) computer readable storage mediums. The data store 920 may be configured to store ePCRs generated at least in part by the patient data charting application 910 and/or the patient data charting application 920a.

In some implementations, the charting system server 918 is configured to interoperate with the CAD system server 930, the navigation system server 928, the billing system server 967, and/or the case data store 924 to acquire patient identification data and/or medical records for patients. In some embodiments, the charting system server 918 is configured to periodically update medical records by interoperating with the other servers in the platform 926 and/or devices within the mobile EMS environment 904. In one example, the charting system server 918 periodically requests updated billing codes from the billing system server 967 and updates medical records stored in the data store 920 accordingly. These billing codes are a source of information for previous medical treatments. For instance, billing codes can indicate that a patient received treatment for asthma, treatment for cardiac arrest, treatment for drug overdose, prescription information, and/or recent surgeries. This information may be clinically actionable and relevant. For example, stitches from recent surgeries could reopen. Devices implanted during surgery may need to be addressed. Treatments for drug overdose may indicate a need to avoid opioids. Repeated treatments and prescriptions could indicate chronic conditions and/or contraindications.

The CAD system server 930, in some embodiments, receives requests to record calls from a public safety answering point and processes the requests to generate and store call records. The CAD system server 930 may transmit dispatch requests to an EMS agency to dispatch EMS personnel (e.g., the care providers 208a-208c of FIG. 2 and/or the EMT 520 of FIG. 5C) to service calls. The CAD system server 930 may transmit addresses to call locations to the mobile EMS environment 904 so that the user interface device 906 (e.g., the patient data charting application 910) and/or the edge server 914 (e.g., the patient data charting application 920a) can acquire routes to call locations by interoperating with the positioning system 940 and/or the navigation system server 928. In some implementations, the user interface device 906 and/or the mobile EMS environment 904 provides real-time, step-by-step directions to call locations via the routes.

In some implementations, the mobile EMS environment 904 includes one or more medical devices 932. The medical devices 932, in some examples, can include a patient treatment device, a patient monitoring device, and/or a combination thereof, for example as described in various examples of the present disclosure. For example, the medical device(s) 932 may include a defibrillator configured to delivery therapeutic electric shocks to the patient, a ventilator configured to delivery ventilation, and/or an automated chest compression device configured to perform automated CPR compressions on a patient. The medical device(s) 932 may further delivery other types of treatment such as, in some examples, operating a respirator and/or administering drugs or other medication. In some examples, the medical devices 932 may include external medical devices, implanted medical devices, and/or insertable medical devices. The medical devices 932 may be external wearable devices, either by caregivers or by patients and/or may include non-wearable devices, like a patient monitor/defibrillator, designed to rest on a floor or a table but not supported by the patient's body. The medical device 932 may be a therapy delivery device, a sensor device, a monitoring device, or a combination thereof. For example, the medical devices 932 may include an external defibrillator (e.g., a patient monitor/defibrillator or an automated external defibrillator (AED)), a trauma kit, an automated compression device, a portable ventilation device, a drug delivery device and/or an ultrasound imaging device. Additionally, the one or more medical devices 932 may include sensors such as, but not limited to, a compression sensor, an SpO2 sensor, a CO2 sensor, a non-invasive blood pressure (NIBP) sensor, electrodes (e.g., sensing electrodes and/or therapy electrodes), an airway pressure sensor, a pneumotachometer, an airflow sensor, a temperature sensor, a Doppler blood flow sensor, an invasive blood pressure (IBP) sensor, a continuous blood pressure sensor, an intubation tube, a mask, a nasal cannula, or a spirometer

The case data store 924, in some implementations, receives case files uploaded by the medical devices 932. The case data store 924 may be implemented by, for example, a database (e.g., a relational database) and stored on one or more non-transitory (non-volatile) computer readable storage mediums. In some embodiments, the case data store 924 includes records that store case data derived from case files from medical devices used to treat patients during encounters. Moreover, in some embodiments, the case data store 924 stores complete copies of the case files themselves (e.g., as large binary objects). The case data stored in the case data store 924 can document patient encounters from the point of view of medical devices, such as the medical devices 932. As such, case data generated by a particular medical device 932 during a patient encounter can include an identifier of the medical device 932, physiologic parameters values of the patient recorded by the medical device 932 during the encounter, characteristics of treatment provided by the medical device 932 to a patient during the encounter, actions taken by care providers during the encounter, and timestamps associated with medical device case data. For instance, where the medical device is a defibrillator, the case data can include patient physiological parameters such as ECG data for the patient, as well as characteristics of therapeutic shocks delivered by the defibrillator to the patient, CPR performance data, and timestamps reflecting a power-on time for the defibrillator and associated with recorded case data, among other information. The patient data charting application 910 and/or the patient data charting application 920a may receive case data from the medical device(s) 932 via the charting system server 918 and/or via short-range communications with the medical device(s) 932. In some embodiments, the patient data charting application 910 and/or the patient data charting application 920a records case data of one or more of the medical device(s) 932, for example by capturing image data of a screen of the medical device(s).

The data stores 920 and 924 can be organized according to a variety of physical and/or logical structures. The data stores 920 and 924 can be organized in a cloud storage database, such as the Google™ Cloud Storage or Amazon™ Elastic File System (EFS™). In some embodiments, the data stores 920 and 924 are implemented within at least one relational database having a highly normalized schema and accessible via a structured query language (SQL) engine, such as Oracle Database management system (DBMS) or Microsoft SQL Server. In some implementations, the platform 926 includes a database querying interface, such as the Google BigQuery™ platform or Amazon RDS™. The schema can, in some embodiments, include columns and tables that enable the data stores 920 and 924 to house data for multiple tenants. In addition, although the description provided above illustrates the data stores 920 and 924 as relational databases, the examples described herein are not limited to that particular physical form. Other databases may include flat files maintained by an operating system and including serialized, proprietary data structures, hierarchical database, xml files, NoSQL databases, document-oriented databases and the like. Thus, the data stores 920 and 924 as described herein are not limited to a particular implementation.

The billing system server 967, in some embodiments, implements a medical billing system. The billing system server 967, for example, can store patient identification data, information regarding claims involving patients, payments status of the claims, and the like. The patient identification data stored in the billing system server 967 can include, for example, patient provider and insurance information.

In combination, the systems illustrated in FIG. 9 can produce accurate and comprehensive documentation that improves continuity of patient care and overall patient health outcomes. More specifically, continuity of care may benefit from a record that thoroughly describes physiological meters and treatments provided through enhanced data collection provided through image analysis of the actions of caregivers during emergency medical procedures.

Reference has been made to illustrations representing methods and systems according to implementations of this disclosure. Aspects thereof may be implemented by computer program instructions. These computer program instructions may be provided to a processor of a general-purpose computer, special purpose computer, or other programmable data processing apparatus and/or distributed processing systems having processing circuitry, such that the instructions, which execute via the processor of the computer or other programmable data processing apparatus, create means for implementing the functions/operations specified in the illustrations.

One or more processors can be utilized to implement various functions and/or algorithms described herein. Additionally, any functions and/or algorithms described herein can be performed upon one or more virtual processors. The virtual processors, for example, may be part of one or more physical computing systems such as a computer farm or a cloud drive.

Aspects of the present disclosure may be implemented by software logic, including machine readable instructions or commands for execution via processing circuitry. The software logic may also be referred to, in some examples, as machine readable code, software code, or programming instructions. The software logic, in certain embodiments, may be coded in runtime-executable commands and/or compiled as a machine-executable program or file. The software logic may be programmed in and/or compiled into a variety of coding languages or formats.

Aspects of the present disclosure may be implemented by hardware logic (where hardware logic naturally also includes any necessary signal wiring, memory elements and such), with such hardware logic able to operate without active software involvement beyond initial system configuration and any subsequent system reconfigurations (e.g., for different object schema dimensions). The hardware logic may be synthesized on a reprogrammable computing chip such as a field programmable gate array (FPGA) or other reconfigurable logic device. In addition, the hardware logic may be hard coded onto a custom microchip, such as an application-specific integrated circuit (ASIC). In other embodiments, software, stored as instructions to a non-transitory computer-readable medium such as a memory device, on-chip integrated memory unit, or other non-transitory computer-readable storage, may be used to perform at least portions of the herein described functionality.

Various aspects of the embodiments disclosed herein are performed on one or more computing devices, such as a laptop computer, tablet computer, mobile phone or other handheld computing device, or one or more servers. Such computing devices include processing circuitry embodied in one or more processors or logic chips, such as a central processing unit (CPU), graphics processing unit (GPU), field programmable gate array (FPGA), application-specific integrated circuit (ASIC), or programmable logic device (PLD). Further, the processing circuitry may be implemented as multiple processors cooperatively working in concert (e.g., in parallel) to perform the instructions of the inventive processes described above.

The process data and instructions used to perform various methods and algorithms derived herein may be stored in non-transitory (i.e., non-volatile) computer-readable medium or memory. The claimed advancements are not limited by the form of the computer-readable media on which the instructions of the inventive processes are stored. For example, the instructions may be stored on CDs, DVDs, in FLASH memory, RAM, ROM, PROM, EPROM, EEPROM, hard disk or any other information processing device with which the computing device communicates, such as a server or computer. The processing circuitry and stored instructions may enable the computing device to perform, in some examples, to process 100 of FIG. 1B, the process 130 of FIG. 1C, the process 150 of FIG. 1D, the method 600 of FIG. 6, the method 700 of FIG. 7A and FIG. 7B, the process 800 of FIG. 8A, the process 820 of FIG. 8B, the process 830 of FIG. 8C, and/or the process 850 of FIG. 8D.

These computer program instructions can direct a computing device or other programmable data processing apparatus to function in a particular manner, such that the instructions stored in the computer-readable medium produce an article of manufacture including instruction means which implement the function/operation specified in the illustrated process flows.

The computing device, in some embodiments, further includes a display controller for interfacing with a display, such as a built-in display or LCD monitor. A general purpose I/O interface of the computing device may interface with a keyboard, a hand-manipulated movement tracked I/O device (e.g., mouse, virtual reality glove, trackball, joystick, etc.), and/or touch screen panel or touch pad on or separate from the display.

Moreover, the present disclosure is not limited to the specific circuit elements described herein, nor is the present disclosure limited to the specific sizing and classification of these elements. For example, the skilled artisan will appreciate that the circuitry described herein may be adapted based on changes in battery sizing and chemistry or based on the requirements of the intended back-up load to be powered.

Although provided for context, in other implementations, methods and logic flows described herein may be performed on modules or hardware not identical to those described. Accordingly, other implementations are within the scope that may be claimed.

While certain embodiments have been described, these embodiments have been presented by way of example only, and are not intended to limit the scope of the present disclosures. Indeed, the novel methods, apparatuses and systems described herein can be embodied in a variety of other forms; furthermore, various omissions, substitutions and changes in the form of the methods, apparatuses and systems described herein can be made without departing from the spirit of the present disclosures. The accompanying claims and their equivalents are intended to cover such forms or modifications as would fall within the scope and spirit of the present disclosures.

1. A patient data charting apparatus for automatically populating electronic patient care record (ePCR) data at an emergency medical scene, the patient data charting apparatus comprising:
a memory configured to store an ePCR comprising a plurality of data fields; and
at least one processor configured to
obtain image data from one or more image capture devices, wherein the image data comprises a sequence of images of performance of a medical procedure by at least one emergency medical services (EMS) caregiver,
analyze the image data to identify a set of steps corresponding to one or more medical procedures, wherein, for each respective step of at least a portion of the set of steps, the analyzing comprises
recognize, within the image data, at least one medical equipment item used during a respective step of the set of steps,
identify, based at least in part on the set of steps, the medical procedure,
responsive to the identification, determine, based at least in part on the medical procedure, at least one first value of at least one first data field of the plurality of data fields, and
populate the at least one first data field of the ePCR with the at least one first value.

2. The patient data charting apparatus of claim 1, wherein:
the at least one processor is further configured to analyze an initial one or more images of the sequence of images to recognize a precursor step performed prior to beginning the medical procedure; and
the precursor step comprises at least one of preparing a given medical equipment item of the at least one medical equipment item, or preparing a site on a patient's body.

3.-4. (canceled)

5. The patient data charting apparatus of claim 1, wherein recognizing the at least one medical equipment item comprises recognizing an identification marking on a given medical equipment item of the at least one medical equipment item, wherein
the identification marking comprises at least one of a particular shape assigned to the given medical equipment item, a particular color assigned to the given medical equipment item, or a machine-readable code.

6.-7. (canceled)

8. The patient data charting apparatus of claim 1, wherein the at least one medical equipment item comprises one or more of gloves, a 3-lead EKG, a 12-lead EKG, a cardiac monitor, a cardioverter, a central intravenous (IV) catheter, an IV bag, a defibrillator, tubing, a ventilator, a bag valve mask, a tourniquet, a splint, a backboard, a cervical collar, a gurney, gauze, an alcohol swab, or a nasal cannula.

9. The patient data charting apparatus of claim 1, wherein the one or more medical procedures comprises one or more of oxygen delivery, intravenous saline delivery, intravenous drug delivery, obtaining a set of vital physiological measurements, cricothyrotomy, nasal intubation, endotracheal intubation, or tourniquet application with infusion.

10.-11. (canceled)

12. The patient data charting apparatus of claim 1, wherein at least one image capture device of the one or more image capture devices comprises a video image capture device.

13. The patient data charting apparatus of claim 12, wherein the video image capture device is a wearable device.

14.-15. (canceled)

16. The patient data charting apparatus of claim 1, wherein each image capture device of the one or more image capture devices is
a) mounted in or on a medical transport vehicle,
b) configured to be held or worn by a given caregiver of the at least one EMS caregiver, or
c) mounted to or integrated into medical equipment.

17.-22. (canceled)

23. The patient data charting apparatus of claim 1, wherein at least one image capture device of the one or more image capture devices is configured to activate image capture based on a detection of an emergency scene activity.

24. The patient data charting apparatus of claim 23, wherein the at least one image capture device is configured to begin capturing the image data responsive to at least one of motion detection or an audible command.

25.-28. (canceled)

29. The patient data charting apparatus of claim 23, wherein:
the at least one processor is further configured to activate the at least one image capture device responsive to detecting arrival at a medical emergency scene; and
detecting the arrival comprises analyzing global positioning system (GPS) signals.

30. (canceled)

31. The patient data charting apparatus of claim 1, wherein:
a patient data charting device comprises the memory and one or more processors of the at least one processor; and
the patient data charting device is embodied in one or more of a smartphone, a tablet, a portable computing device, a wearable computing device, or combinations thereof.

32.-44. (canceled)

45. The patient data charting apparatus of claim 1, wherein:
an organizational structure of the ePCR comprises data field sections organized according to medical procedure categories; and
the data field sections comprise one or more of a respiratory section or a cardiac section.

46. (canceled)

47. The patient data charting apparatus of claim 1, wherein the at least one processor is further configured to:
identify at least one second data field of the plurality of data fields as having a procedural relationship with the at least one first data field; and
identify, based on the image data, at least one second value of the at least one second data field.

48.-59. (canceled)

60. The patient data charting apparatus of claim 1, wherein:
the at least one processor is further configured to identify, by analyzing the image data, one or more caregivers of the at least one EMS caregiver, wherein analyzing the image data comprises at least one of
i) identifying, within the image data, a caregiver identification badge, or
ii) performing facial recognition on a portion of the image data to obtain facial recognition metrics and matching the facial recognition metrics to a particular caregiver of the at least one EMS caregiver.

61.-62. (canceled)

63. The patient data charting apparatus of claim 60, wherein recognizing the medical procedure comprises recognizing performance of at least a portion of a set of steps of the medical procedure by a particular caregiver of the at least one EMS caregiver.

64. The patient data charting apparatus of claim 63, wherein;
the at least one processor is further configured to determine, using an identification of the particular caregiver, a certification level, an employee level, and/or skill level of the particular caregiver; and
recognizing the medical procedure comprises matching the medical procedure to the certification level, the employee level, and/or the skill level of the particular caregiver.

65.-80. (canceled)

81. The patient data charting apparatus of claim 1, further comprising a network interface coupled to the at least one processor and configured to communicably couple to at least one separate computing device via a network, wherein
the at least one separate computing device comprises a first image capture device of the one or more image capture devices.

82.-87. (canceled)

88. The patient data charting apparatus of claim 1, wherein the at least one processor is further configured to determine, responsive to analyzing the image data, at least one of success or failure of the medical procedure.

89. The patient data charting apparatus of claim 88, wherein determining the at least one of success or failure of the medical procedure comprises calculating at least one timing of the medical procedure, wherein
a first timing of the at least one timing corresponds to a length of time maintaining a piece of medical equipment in a position of therapeutic use.

90. (canceled)

91. The patient data charting apparatus of claim 88, wherein determining the at least one of success or failure of the medical procedure comprises at least one of:
a) recognizing removal of at least one piece of medical equipment;
b) recognizing an end point of the set of steps; or
c) recognizing repetition of at least a portion of the set of steps.

92.-95. (canceled)

96. The patient data charting apparatus of claim 1, further comprising at least one image capture device of the one or more image capture devices.

97.-169. (canceled)
